(19) United States
(12) Patent Application Publication
(10) Pub. No.: US 2023/0330426 A1
(43) Pub. Date: Oct. 19, 2023

(54) IMPLANTATION TECHNIQUES FOR ELECTRIC FIELD THERAPY

(71) Applicant: Medtronic, Inc., Minneapolis, MN (US)

(72) Inventors: Steven M. Goetz, North Oaks, MN (US); Lisa M. Jungbauer Nikolas, Lino Lakes, MN (US); Benjamin Kevin Hendricks, Phoenix, AZ (US); Robert L. Olson, White Bear Lake, MN (US); Richard T. Stone, Roseville, MN (US)

(21) Appl. No.: 18/300,899

(22) Filed: Apr. 14, 2023

Related U.S. Application Data
(60) Provisional application No. 63/331,807, filed on Apr. 16, 2022.

(51) Int. Cl. A61N 1/05; A61N 1/40

(52) U.S. Cl. CPC A61N 1/40; A61N 1/0526; A61N 1/3787

(57) ABSTRACT

Devices, systems, and techniques are disclosed for delivering electric field therapy to tissue of a subject. In one example, a technique includes removing a tumor from tissue to create a resection cavity, implanting a plurality of leads into the tissue adjacent to the resection cavity, and affixing the plurality of leads at least one of the tissue or bone. The example technique also includes tunneling proximal ends of the leads to an implantation pocket and coupling the proximal ends of the leads to an implantable medical device configured to be placed within the implantation pocket, wherein the implantable medical is configured to deliver electric field therapy via the plurality of implantable leads disposed adjacent to the resection cavity.

This application claims the benefit of U.S. Provisional Pat. Application No. 63/331,807, filed Apr. 16, 2022, the entire contents of which is incorporated herein by reference.

### TECHNICAL FIELD

This disclosure generally relates to implantation techniques and devices for electric field therapy.

### BACKGROUND

Alternating electric field (AEF) therapy, is a type of electric field therapy which uses low-intensity electrical fields to treat brain tumors; glioblastoma in particular. Conventional cancer treatments include chemotherapy and radiation, which are associated with treatment-related toxicity and high rates of tumor recurrence. AEF uses an alternating electric field to disrupt cell division in cancer cells, thereby inhibiting cellular replication and initiating apoptosis (cell death). AEF therapy is typically delivered via electrodes located external to the patient.

### SUMMARY

In general, the disclosure describes devices, systems, and techniques related to delivering electric and magnetic stimulation therapy, which includes electrical field therapy and/or detecting electrical signals. Electric field therapy may include modulated electrical field therapy which may include types of electrical field modulation, such as alternating current stimulation which includes alternating electrical field (AEF) therapy and is discussed herein as one example of therapy. Other types of electric and magnetic stimulation therapy are described herein in various examples and combinations. For example, an implantable medical device (IMD) may be coupled to one or more leads carrying one or more electrodes. The IMD may alternate delivery of electrical fields from respective different electrode combinations utilizing some or all of the implanted electrodes carried on the one or more leads.

The one or more leads may be configured to position the array of electrodes with respect to target tissue that is intended to receive the electrical field modulation. For example, the leads may include one or more structures configured to dispose the electrodes at a desired location with respect to a target tissue and/or tissue associated with a tissue resection region. These leads may include cylindrical leads, paddle leads, or other leads that carry one or more electrodes. These leads may be implanted within the brain and around or partially within a resection cavity from which a tumor has been removed. In some examples, one or more implantation plates may be configured to assist a surgeon in implanting the leads to the desired location with respect to the resection cavity and/or fix the leads in place. Each electrode may be independently controllable, or some electrodes of leads or entire leads may be electrically coupled together such that the IMD controls groups or subsets of the total number of electrodes carried by the implanted leads.

The tissue resection region may be a region within the anatomy of the patient where tissue was removed, such tissue that included tumor cells, e.g., glioblastomas or other types of tumor or cancer cells. The remaining cells in or near the tissue resection region may thus be treated by the electrical field modulation via the electrodes of the leads. For example, AEF therapy may be used for various reasons, such as reducing or preventing the growth of tumor cells, such as glioblastomas, or the reduction in growth or proliferation or directional migration of non-tumorous cells within the body. Examples of cells may be within the following tissues: skin, muscle, pulmonary, laryngeal, nasopharyngeal, liver, gastric, splenic, renal, intestinal, pancreatic, or prostate. These manipulations of normal cells could be conducted to address pathological processes or to enhance efficiency of normal cellular functions, such as secretory, migratory, or differentiational activities. AEF therapy has been demonstrated to impact the microstructural elements within cells (e.g., microtubules and/or actin filaments) such that a system can precisely deliver AEF therapy to a subpopulation of cells in a targeted manner to direct or restrict cell migratory activities. In some examples, AEF therapy may be delivered to a patient to modulate fibroblasts and their role in scar tissue formation or modulate the proliferation of lymphocytes or leukocytes for patients with an auto-immune condition.

In one example, a system includes a memory configured to store parameters defining electric field therapy, and processing circuitry configured to control an implantable medical device to deliver electric field therapy according to the parameters by at least alternating electric fields between electrodes of adjacent lead pairs of a plurality of implantable leads disposed adjacent to a resection cavity of a patient.

In another example, a method includes controlling, by processing circuitry, an implantable medical device to deliver electric field therapy according to the parameters defining the electric field therapy by at least alternating electric fields between electrodes of adjacent lead pairs of a plurality of implantable leads disposed adjacent to a resection cavity of a patient.

In another example, a non-transitory computer-readable storage medium including instructions that, when executed, cause processing circuitry to control an implantable medical device to deliver electric field therapy according to the parameters defining the electric field therapy by at least alternating electric fields between electrodes of adjacent lead pairs of a plurality of implantable leads disposed adjacent to a resection cavity of a patient.

In another example, a system includes a plurality of implantable leads configured to be disposed within tissue adjacent a resection cavity of a patient; and an implantable medical device configured to deliver electric field therapy via the plurality of implantable leads disposed adjacent to the resection cavity.

In another example, a method includes removing a tumor from tissue to create a resection cavity, implanting a plurality of leads into the tissue adjacent to the resection cavity, affixing the plurality of leads at least one of the tissue or bone, tunneling proximal ends of the leads to an implantation pocket, and coupling the proximal ends of the leads to an implantable medical device configured to be placed within the implantation pocket, wherein the implantable medical is configured to deliver electric field therapy via the plurality of implantable leads disposed adjacent to the resection cavity.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver alternating electric field (AEF) therapy to a patient according to an example of the techniques of the disclosure.

FIG. 2 is a block diagram of the example IMD of FIG. 1 for delivering AEF therapy according to an example of the techniques of the disclosure.

FIG. 3 is a block diagram of the external programmer of FIG. 1 for controlling delivery of AEF therapy according to an example of the techniques of the disclosure.

FIG. 4 is a block diagram illustrating an example system that includes an external device, such as a server, and one or more computing devices that are coupled to an implantable medical device and external programmer shown in FIG. 1 via a network.

FIGS. 5A and 5B are conceptual diagrams of example leads with respective electrodes carried by the lead.

FIGS. 5C, 5D, 5E, and 5F are conceptual diagrams of example electrodes disposed around a perimeter of a lead at a particular longitudinal location.

FIG. 6 is a flowchart illustrating an example technique for delivering AEF therapy to a patient.

FIG. 7 is a flowchart illustrating an example technique for implanting multiple leads for delivering AEF therapy to a patient.

FIG. 8 is a conceptual diagram of example electrical fields generated between implanted leads.

FIG. 9 is a conceptual diagram illustrating example leads implanted around a resection cavity to deliver alternating magnetic field (AEF) therapy.

FIGS. 10A-10F are conceptual diagrams illustrating example lead configurations and corresponding electric fields between the leads.

FIGS. 11A and 11B are illustrations of an example set of implantable leads implanted with respect to a resection cavity.

FIG. 11C is a conceptual diagram of an example fixation structure for securing leads within tissue.

FIG. 12 is an example system that includes a lead and spacer that facilitates implantation.

FIGS. 13A and 13B are conceptual diagrams illustrating example systems for powering the delivery of AEF therapy.

FIG. 14 is a conceptual diagram illustrating an example system powering the delivery of AEF therapy.

FIG. 15 is a cross-sectional view of an implantation plate for implanting leads around a resection cavity.

FIGS. 16A and 16B are top views of example implantation plates for implanting leads around a resection cavity.

FIG. 17 is a cross-sectional view of an implantation plate and inflatable member configured to fill a resection cavity during lead implantation.

FIG. 18 is a conceptual diagram of an example system that includes an implantable medical device and multiplexer to control AEF therapy.

FIG. 19 is a conceptual diagram of example alternating electric fields between an example array of leads.

FIG. 20 is a conceptual diagram of an example paddle lead configured to be cut to a desired size for a patient.

### DETAILED DESCRIPTION

This disclosure describes various devices, systems, and techniques for delivering modulated electrical field therapy (which may include the example of AEF therapy) to a patient via implanted electrodes. Alternating electric field application is a cancer treatment type with the potential to reduce treatment related toxicity. In alternating electric field application, an alternating electric field is applied to a cancerous region of the brain, which may disrupt cellular division for rapidly-dividing cancer cells. To administer alternating electric field treatment to a patient, an external system can be applied near the anatomy of interest, such as around the cranium of the patient, in order to deliver the alternating electric field to the patient. However, there are various challenges to external delivery of AEF therapy. For example, external AEF therapy requires that hair be removed from the scalp of the patient. As another example, electrical fields delivered from the external electrodes for extended periods of time required for AEF therapy may cause increased tissue heating and potential burns on the skin of the patient. In addition, external electrodes may prevent localized treatment of tumors within the brain. For implanted electrodes, it may be difficult to implant leads at desired locations of tumor cells, such as tumor cells that may remain after tissue resection. In addition, there are challenges related to treating all possible tissue that could include tumor cells around the resection cavity.

As described herein, a system may include one or more leads configured to deliver electric field therapy (also referred to as AEF therapy in some examples) from implanted electrodes at a location and strength specific for the patient. Electric field therapy may generally refer to therapy in which electrical fields are modulated to provide some therapeutic response. For example, alternating electric field therapy described herein includes a system that modulates electric fields by alternating between different electrode combinations, different field directions, and/or other parameters that define the electric field therapy). This internal AEF therapy may act to inhibit cellular division and/or initiate apoptosis of cancer cells at the targeted treatment location. The implanted electrodes or lead configuration within tissue may be selected to target tissue identified as including cancerous cells or tissue around a resection area (e.g., a tissue resection region) where a previous tumor was removed. Example systems described herein may provide AEF or other electric field therapy using relatively simple cylindrical leads and electrodes, and may include multiplexing of electrical signals to use a plurality of different electric fields and/or different therapies. Some leads or electrodes may be configured for sensing signals in order to identify electric fields and/or physiological signals.

For example, a plurality of cylindrical leads may be inserted into the resection area adjacent to the resection cavity. Each of the cylindrical leads may include a plurality of electrodes, such as 2, 4, or 8 electrodes each. These cylindrical leads may be spaced out around the perimeter of the resection cavity in order to target any tumor cells that may remain after the resection. In some examples, one or more implantation plates and/or other devices may be employed by the clinician in order to aid in the insertion and/or retention of the leads at the target location in the resection region. These implantation plates or other devices may be helpful since the resection region of tissue may begin to collapse after resection which reduces the likelihood that leads can be inserted or remain in the target location.

In this manner, the system may operate to deliver AEF therapy to reduce cancerous cells in the patient and/or prevent or reduce the reoccurrence of cancer after resection. A computing device may be used for planning implantation of electrodes and/or selection of the number of leads and lead placement or other stimulation parameters based on imaging data obtained for the patient used to generate a model of patient tissue. The AEF therapy described herein may facilitate patient-specific AEF therapy directed to specific target tissue. Using implanted electrodes may enable the system to operate over larger periods of time without impacting most patient daily activities. In addition, leads using multiple cylindrical leads placed in tissue adjacent the resection cavity may be an effective method to place electrodes for creating desired electric fields and may reduce tissue damage during implantation and/or reduce the surgical time needed for a clinician to implant the lead within the patient. These and other advantages may be realized by the systems and examples described herein.

Although this disclosure is directed to delivery of AEF therapy to the brain for the purpose of treating glioblastoma, the systems, devices, and techniques described herein may similarly operate to deliver AEF therapy or similar electric-field therapies to other tissue areas and/or to treat different types of cancer. For example, a system may be implanted to treat and/or prevent cancer in the spine, pelvis, abdomen, or any other location. Some examples of target tissue may include regions of expected metastatic elements, such as lymph nodes, to reduce the spread of cells from a different tumor cite. Moreover, a human patient is described for example purposes herein, but similar systems, devices, and techniques may be used for other animals in other examples.

Electric field therapy described herein may include several different types of therapy in which different electric fields are delivered to a patient. These therapies may include modified electric field therapy, modulated electric field therapy, alternating electric field (AEF) therapy, or other therapies in which different electric fields are delivered to a patient. In some examples, these different electric fields change over time in a symmetric, non-symmetric, continuous, and/or non-continuous manner. While reference is primarily made to AEF in the examples described herein, other types of electric field therapy can be applied in the various example devices, systems, and techniques described herein.

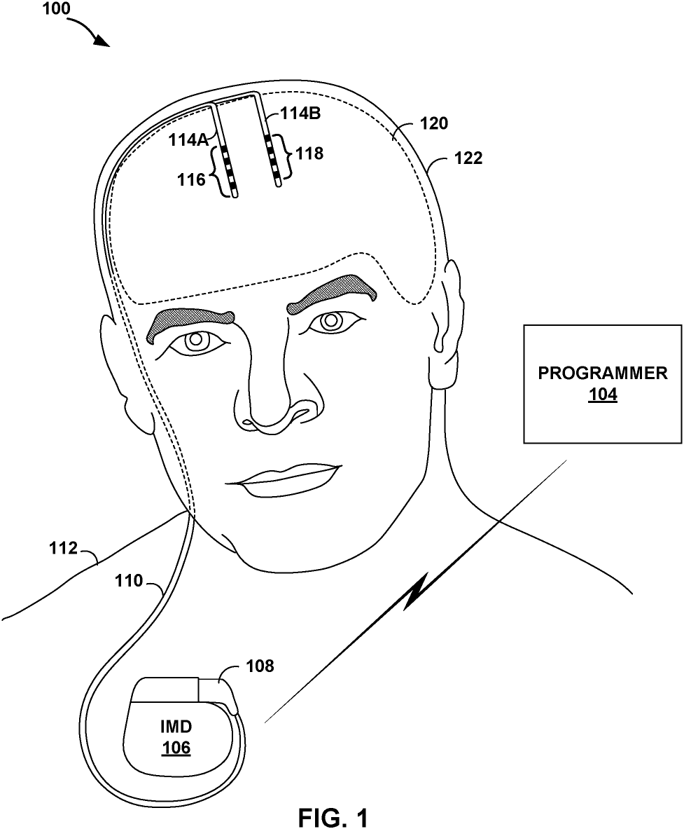

FIG. 1 is a conceptual diagram illustrating an example system 100 that includes an implantable medical device (IMD) 106 configured to deliver therapy to patient 112 according to an example of the techniques of the disclosure. This therapy may be AEF therapy or another therapy based on applied electrical fields. As shown in the example of FIG. 1, example system 100 includes medical device programmer 104, implantable medical device (IMD) 106, lead extension 110, and leads 114A and 114B with respective sets of electrodes 116, 118. In the example shown in FIG. 1, electrodes 116, 118 of leads 114A, 114B are positioned to deliver electrical stimulation to a tissue site within brain 120, such as a deep brain site under the dura mater of brain 120 of patient 112. In some examples, delivery of electric fields (e.g., electrical stimulation) to one or more regions of brain 120, such as a region that contains a tumor such as glioblastoma, or region from which a glioblastoma was resected (removed). This location where the tumor was removed, e.g., the tumor bed, may be or be part of the target tissue for AEF therapy. The tumor bed may be of various sizes, but may be between approximately 1 mm to 3 mm in diameter in some examples. Some or all of electrodes 116, 118 also may be positioned to sense neurological brain signals within brain 120 of patient 112. In some examples, some of electrodes 116, 118 may be configured to sense neurological brain signals, impedance, etc., and some or all of electrodes 116, 118 may be configured to deliver electrical stimulation to brain 120 in the form of AEF therapy. In other examples, all of electrodes 116, 118 are configured to both sense electrical signals and deliver electrical stimulation to brain 120. Leads 114A, 114B are cylindrical leads and are merely examples, as any other leads or lead configurations described herein may be configured to position respective electrodes within brain 120 to deliver AEF therapy to patient 122. For example, four, six, or more leads similar to leads 114A and 114B may be implanted in other examples.

IMD 106 includes a therapy module (e.g., which may include processing circuitry, signal generation circuitry or other electrical circuitry configured to perform the functions attributed to IMD 106) that includes a stimulation generator configured to generate and deliver electrical stimulation therapy (e.g., AEF therapy) to patient 112 via a subset of electrodes 116, 118 of leads 114A and 114B, respectively. The subset of electrodes 116, 118 that are used to deliver electrical stimulation to patient 112, and, in some cases, the polarity of the subset of electrodes 116, 118, may be referred to as a stimulation electrode combination. As described in further detail below, the stimulation electrode combination can be selected for a particular patient 112 and target tissue site (e.g., selected based on the patient condition or based on the determined location of a tumor or other tissue of interest). The group of electrodes 116, 118 includes at least one electrode and can include a plurality of electrodes.

In some examples, the plurality of electrodes 116 and/or 118 may have a complex electrode geometry such that two or more electrodes of the lead are located at different positions around the perimeter of the respective lead (e.g., different positions around a longitudinal axis of the lead). In other examples, the electrodes at different positions around the perimeter of the lead may be disposed on different structures of the lead. In this manner, electrodes at different perimeter locations may be used to generate different electrical fields. For example, anodes on a first side of a first lead and cathodes on a second side of a second lead, wherein the first sides and second side face opposing directions, may be used to generate a first electric field. The second electric field may be generated with cathodes on the second side of the first lead and anodes on the first side of the second lead. In this manner, alternating between the first and second electric fields may generate electrical current that changes the polarities of cellular components to disrupt cell division. Although two leads 14 are shown in the example of FIG. 1, a single lead, three leads, four leads, five leads, or more leads may be implanted in different examples. In any case, the combination of leads may provide an overall array of electrodes that can be programmed to deliver alternating electrical fields to a target tissue. These complex electrode geometries can also enable directional sensing that can measure the orientation of electric fields generated in tissue. For example, the system may measure electrical potentials between electrodes at different locations on a lead or between different leads to determine a gradient of electrical potentials and a gradient of the delivered electrical field. The system can then determine electric field spread and configure the electric fields and/or calibrate a predictive model of field spread based on the sensed gradient of electrical potentials.

In some examples, the neurological signals (e.g., an example type of electrical signals) sensed within brain 120 may reflect changes in electrical current produced by the sum of electrical potential differences across brain tissue. Examples of neurological brain signals include, but are not limited to, electrical signals generated from local field potentials (LFP) sensed within one or more regions of brain 120, electroencephalogram (EEG) signals, or electrocorticogram (ECoG) signals. Any of these sensed signals may be intrinsic signals generated by physiological neural activity and/or evoked signals generated in response to a delivered stimulus (e.g., a delivered electrical stimulation signal). It is noted that modulated electric field therapy (e.g., AEF therapy) may not evoke neuron propagation or affect other normal neurological function. However, the system may deliver signals intended to affect neurological processes in order to sense signals that may be indicative of physiological states or the response to modulated electric field therapy. In some examples, the system may utilize any electrode combinations to directly sense the electrical field (e.g., field strengths, field locations, or other characteristics) delivered by other electrode combinations. In this manner, the system may confirm expected electrical field strengths, adjust one or more stimulation parameters that define the electrical fields to effect target tissue (e.g., to match a desired stimulation model), and/or adjust the model of stimulation to reflect the reality of tissue characteristics. In some examples, the system may adjust the stimulation parameters defining the electrical fields to accommodate for tissue changes over time and/or lead movement within the patient after surgery or over time. The system may adjust any of these parameters in response to reviewing previously stored data and/or in real-time as sensed data is received or generated.

In some examples, the neurological brain signals that are used to select a stimulation electrode combination may be sensed within the same region of brain 120 as the target tissue site for the electrical stimulation and/or from a region different (e.g., adjacent to or outside of) than the target tissue site. The system may be configured to compute or predict the electrical field at the target tissue based on the signals sensed within the target tissue and/or at a region different than the target tissue. The specific target tissue sites and/or regions within brain 120 may be selected based on the patient condition or location, size, depth, and/or volume of a tumor or resection bed. In addition, the system may recommend a certain number and/or type of leads to be implanted as well as a desired location, angle of entry, and spatial distance between each lead. Thus, due to these differences in target locations, in some examples, the electrodes used for delivering electrical stimulation may be different than the electrodes used for sensing neurological brain signals. In other examples, the same electrodes may be used to deliver electrical stimulation and sense brain signals. However, this configuration of using the same electrodes could require the system to switch between stimulation generation and sensing circuitry and may reduce the time the system can sense brain signals. In some examples, the system may be configured to deliver electrical signals to generate the electrical fields from the same electrode configurations (or using at least some of the same electrodes) in an at least partially interleaved basis.

Electrical stimulation generated by IMD 106 may be configured to manage a variety of disorders and conditions. In some examples, the stimulation generator of IMD 106 is configured to generate and deliver electrical stimulation pulses to patient 112 for AEF therapy via electrodes of a selected stimulation electrode combination. However, in other examples, the stimulation generator of IMD 106 may be configured to generate and deliver a continuous wave signal, e.g., a sine wave or triangle wave of specified amplitude (peak to peak) and frequency as part of the electrical fields of the AEF therapy. Generally, modulated electric field therapy (e.g., AEF therapy) may include the delivery of the continuous wave signal(s), but the waveforms may be symmetric, asymmetric, non-continuous, continuous, cycled, interleaved between different combinations, constant, or otherwise changing over time at random or predetermined sequences. In either case, a stimulation generator within IMD 106 may generate the AEF therapy according to a therapy program that is selected at that given time in therapy. In examples in which IMD 106 delivers electrical stimulation in the form of stimulation pulses, a therapy program may include a set of therapy parameter values (e.g., stimulation parameters), such as a stimulation electrode combination for delivering different electrical fields to patient 112, pulse frequency, pulse width, and a current or voltage amplitude of the pulses or continuous signals. As previously indicated, the electrode combination may indicate the specific electrodes 116, 118 that are selected to deliver stimulation signals to tissue of patient 112 and the respective polarities of the selected electrodes. IMD 106 may deliver electrical stimulation intended to contribute to a therapeutic effect. In some examples, IMD 106 may also, or alternatively, deliver electrical stimulation intended to be sensed by other electrodes and/or elicit a physiological response, such as an evoked compound action potential (ECAP), that can be sensed by electrodes. The delivered stimulation may have a sub-perception threshold intensity or supra-perception threshold intensity. In this manner, IMD 106 may employ sensed electrical stimulation and/or sensed physiological responses in a closed-loop manner to modulate delivered electrical stimulation, such as AEF therapy.

IMD 106 may be implanted within a subcutaneous pocket above the clavicle, or, alternatively, on or within cranium 122 or at any other suitable site within patient 112 such as a lower abdominal or high buttock location. Other configurations might include IMD 106 implanted at multiple locations, such as near a site of tumor occurrence and remote sites of likely tumor transmission or spread. Generally, IMD 106 is constructed of a biocompatible material that resists corrosion and degradation from bodily fluids. IMD 106 may comprise a hermetic housing to substantially enclose components, such as a processor, therapy module, and memory. Other implant locations for IMD 106 may be utilized for treatment of the brain or other tissues. Example alternative implantation sites for IMD 106 may include the lower back, shoulder, neck, abdomen, or any other location.

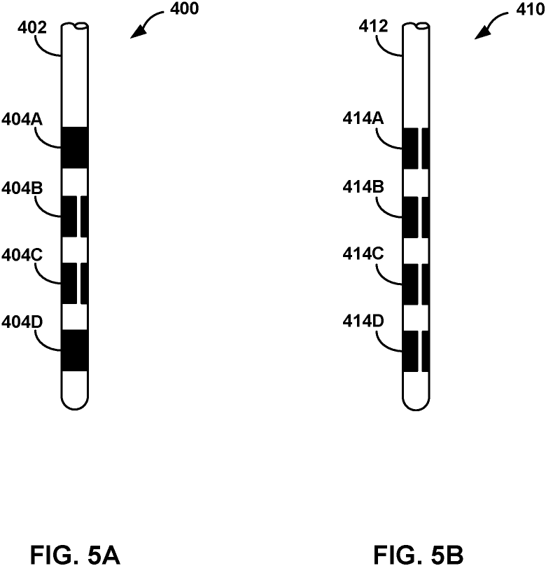

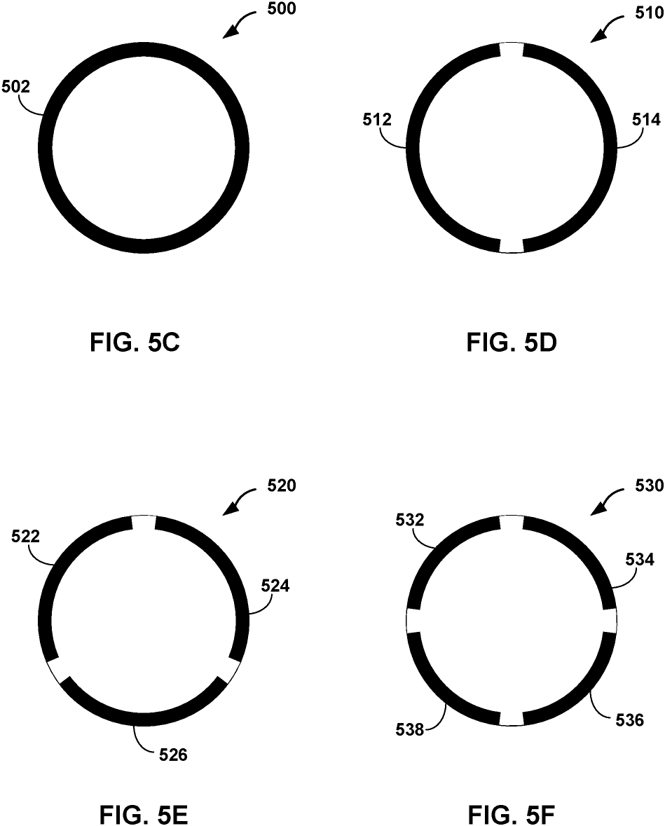

As shown in FIG. 1, implanted lead extension 110 is coupled to leads 114A and 114B and IMD 106 via connector 108 (also referred to as a connector block or a header of IMD 106). In the example of FIG. 1, lead extension 110 traverses from the implant site of IMD 106 and along the neck of patient 112 to cranium 122 of patient 112 to access brain 120. In the example shown in FIG. 1, leads 114A and 114B (collectively “leads 114”) are implanted within the right and left hemispheres, respectively, of patient 112 in order deliver AEF therapy to one or more regions of brain 120, which may be selected based on the patient condition or disorder controlled by therapy system 100. The specific target tissue site and the stimulation electrodes used to deliver stimulation to the target tissue site, however, may be selected, e.g., according to the locations of a tumor or resection bed and/or other sensed patient parameters. Other lead 114 and IMD 106 implant sites are contemplated. For example, IMD 106 may be implanted on or within cranium 122, in some examples. Or leads 114 may be implanted within the same hemisphere or IMD 106 may be coupled to a single lead implanted in a single hemisphere. Although leads 114 may have ring electrodes at different longitudinal positions as shown in FIG. 1 (e.g., cylindrical leads), leads 114 may have electrodes disposed at different positions around the perimeter of the lead (e.g., different circumferential positions for a cylindrical shaped lead) as shown in the examples of FIGS. 5A and 5B.

Leads 114 illustrate an example lead set that include axial leads carrying ring electrodes disposed at different axial positions (or longitudinal positions). In other examples, leads may be referred to as “paddle” leads carrying planar arrays of electrodes on one side of the lead structure or a “grid” of electrodes that enable the placement of electrical elements at a variety of locations around the tissue. In addition, as described herein, complex lead array geometries may be used in which electrodes are disposed at different respective longitudinal positions and different positions around the perimeter of the lead. For example, a lead 114 may include a lead housing (e.g., a structure configured to housing conductors that travel from a proximal end to a distal end of the lead) and one or more structures coupled to the housing. Lead 114 may also include a plurality of electrodes disposed on the one or more structures and configured to deliver electrical fields to tissue. These one or more structures may be configured to position the plurality of electrodes with respect to a tissue resection region or other target tissue that is intended to receive the electrical fields (e.g., AEF therapy). For example, one or more curved sheaths may be placed over at least a portion of one or both of leads 114 to position the electrodes in a curved or non-linear arrangement. In this manner, lead 114 may be configured to be implanted within a patient comprising the tissue resection region.

Although leads 114 are shown in FIG. 1 as being coupled to a common lead extension 110, in other examples, leads 114 may be coupled to IMD 106 via separate lead extensions or directly to connector 108. Leads 114 may be positioned to deliver electrical stimulation to one or more target tissue sites within brain 120. Leads 114 may be implanted to position electrodes 116, 118 at desired locations of brain 120 through respective holes, or a common hole, in cranium 122. Leads 114 may be placed at any location within brain 120 such that electrodes 116, 118 are capable of providing electrical stimulation to target tissue sites within brain 120 during treatment. For example, electrodes 116, 118 may be surgically implanted under the dura mater of brain 120 or within the cerebral cortex of brain 120 via a burr hole in cranium 122 of patient 112, and electrically coupled to IMD 106 via one or more leads 114.

In the example shown in FIG. 1, electrodes 116, 118 of leads 114 are shown as ring electrodes. Ring electrodes may be used in AEF therapy applications because they are relatively simple to program and are capable of delivering an electrical field to any tissue adjacent to electrodes 116, 118. In other examples, electrodes 116, 118 may have different configurations. For example, in some examples, at least some of the electrodes 116, 118 of leads 114 may have a complex electrode array geometry that is capable of producing electrical fields of various shapes and electrical fields directed to different directions with respect to the lead, such as in the various medical leads described herein. The complex electrode array geometry may include multiple electrodes (e.g., partial ring or segmented electrodes) around the outer perimeter of each lead 114, rather than one ring electrode, such as shown in FIGS. 5A and 5B. In this manner, electrical stimulation may be directed in a specific direction, such as alternating directions for alternating electrical fields, from leads 114 to provide AEF therapy. In some examples, one or more leads 114 may include insulation on a portion of the lead that may enable electrical field directionality such that the electrical current is directed to certain circumferential locations other than the insulated portion. In some examples, fewer electrodes may be used to generate smaller electrical fields specifically selected to affect target tissue during the AEF delivery while avoiding subjecting other tissues to the electric fields. In some examples, a housing of IMD 106 may include one or more stimulation and/or sensing electrodes. In alternative examples, leads 114 may have shapes other than elongated cylinders as shown in FIG. 1. For example, leads 114 may be paddle leads, spherical leads, bendable leads, leads having one or more structures that extend and/or expand from a lead housing, or any other type of shape that positions electrodes to be effective in treating patient 112 and/or minimizing invasiveness of leads 114. In this manner, any electrode arrays may be designed to be placed surgically in a tumor void or bed adjacent to the resection cavity and deliver electric fields to cover the tissue adjacent the interior volume of the debulked void therein.

In the example shown in FIG. 1, IMD 106 includes a memory to store a plurality of therapy programs that each define a set of therapy parameter values. In some examples, IMD 106 may select a therapy program from the memory based on various parameters, such as sensed patient parameters and the identified patient behaviors. IMD 106 may generate electrical or magnetic stimulation based on the selected therapy program to deliver effective AEF therapy that reduces or prevents cancerous cell division, enhances apoptosis of cancer cells, facilitates immune-mediated cell death, or modulates other cellular functions, such as cell differentiation or de-differentiation, or secretory vesicle release. In other examples, AEF therapy may be delivered for additional or alternative benefits. For example, the system may target AEF therapy to fibroblasts in order to inhibit scar formation within a wound. For a patient with an auto-immune disease, the system may deliver AEF therapy to lymphatic channels, the spleen, thymus, or other anatomical location within the patient to modulate the proliferation of lymphocytes or leukocytes.

External programmer 104 wirelessly communicates with IMD 106 as needed to provide or retrieve therapy information. Programmer 104 is an external computing device that the user, e.g., a clinician and/or patient 112, may use to communicate with IMD 106. For example, programmer 104 may be a clinician programmer that the clinician uses to communicate with IMD 106 and program one or more therapy programs for IMD 106. Alternatively, programmer 104 may be a patient programmer that allows patient 112 to select programs and/or view and modify therapy parameters. The clinician programmer may include more programming features than the patient programmer. In other words, more complex or sensitive tasks may only be allowed by the clinician programmer to prevent an untrained patient from making undesirable changes to IMD 106. IMD 106 may also transmit notifications to programmer 104 for delivery to a user in response to detecting one or more problems with stimulation and/or detection of one or more trigger events for patient 112. Programmer 104 may enter a new programming session for the user to select new stimulation parameters for subsequent therapy. External programmer 104 may display estimated locations of target tissue locations and/or suggested stimulation parameter values for delivering electrical stimulation that affects the target tissue location.

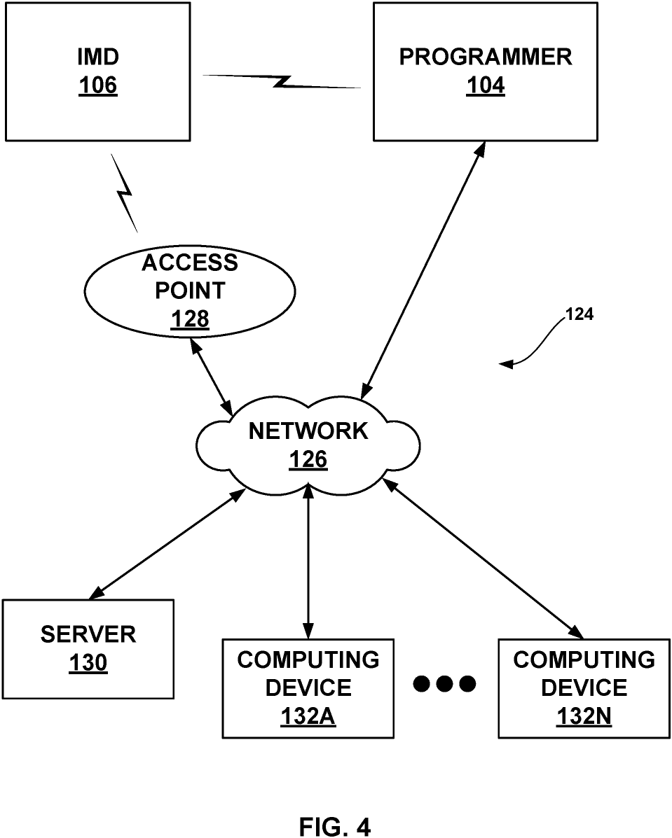

When programmer 104 is configured for use by the clinician, programmer 104 may be used to transmit initial programming information to IMD 106. This initial information may include hardware information, such as the type of leads 114 and the electrode arrangement, the position of leads 114 within brain 120, the configuration of electrode array 116, 118, initial programs defining therapy parameter values, and any other information the clinician desires to program into IMD 106. Programmer 104 may also be capable of completing functional tests (e.g., measuring the impedance of electrodes 116, 118 of leads 114 or the electric field strength at a strategic location on one of leads 114). In some examples, programmer 104 may receive sensed signals or representative information and perform the same techniques and functions attributed to IMD 106 herein. In other examples, a remote server (e.g., a standalone server or part of a cloud service as shown in FIG. 4) may perform the functions attributed to IMD 106, programmer 104, or any other devices described herein.

Programmer 104 may also be configured for use by patient 112. When configured as a patient programmer, programmer 104 may have limited functionality (compared to a clinician programmer) in order to prevent patient 112 from altering critical functions of IMD 106 or applications that may be detrimental to patient 112. In this manner, programmer 104 may only allow patient 112 to adjust values for certain therapy parameters or set an available range of values for a particular therapy parameter. In one example, a patient programmer may only allow for functions such as turning AEF therapy on or off and/or decreasing stimulation intensity. In some examples, programmer 104 may present an indication of delivery time for the patient, such as a screen that indicates the amount of time that the therapy is delivered and/or time the therapy has been off for each day, week, month, etc. For example, programmer 104 may present that “AEF therapy has been delivered for 85% of the time during the last week” or “AEF therapy has been delivered during 6 of the last 7 days.” In addition, programmer 104 may present remaining therapy time available before recharge is required when IMD 106 operates using a rechargeable power source. In some examples, the user interface may present a map of the cranium with the electrode configuration represented and one or more zones of tissue that receive a specified therapeutic parameter (such as V/cm). In some examples, the user interface may be configured to receive user input manipulating the orientation of the field and/or adjustment of other stimulation parameter values.

Programmer 104 may also provide an indication to patient 112 when therapy is being delivered, when patient input has triggered a change in therapy or when the power source within programmer 104 or IMD 106 needs to be replaced or recharged. For example, programmer 112 may include an alert LED, may flash a message to patient 112 via a programmer display, generate an audible sound or somatosensory cue to confirm patient input was received, e.g., to indicate a patient state or to manually modify a therapy parameter.

Therapy system 100 may be implemented to provide chronic stimulation therapy to patient 112 over the course of several months or years. However, system 100 may also be employed on a trial basis to evaluate therapy before committing to full implantation. For example, leads 114 may be configured to be implanted for a relatively short time (e.g., a few weeks or months) or for longer periods of years for chronic use. If implemented temporarily, some components of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates AEF system 100 provides effective treatment to patient 112, the clinician may implant a chronic stimulator within patient 112 for relatively long-term treatment with AEF therapy.

Although IMD 106 is described as delivering electrical stimulation therapy to brain 120, IMD 106 may be configured to alternatively, or additionally, direct electrical stimulation to other anatomical regions of patient 112 in other examples. In other examples, system 100 may include an implantable drug pump in addition to, or in place of, IMD 106. Further, an IMD may provide other electrical stimulation such as spinal cord stimulation to treat other types of cancer or other diseases or disorders. In some embodiments, the therapy delivered by IMD 106 is designed to enhance the ability of particular drugs to pass through the blood-brain barrier, or is designed to enable particular drugs to pass through the blood-brain barrier, through for example, delivering therapy at a specific parameter set such as a frequency of 100 kHz. The therapy may function by either specification of opening diameter within the blood brain barrier or by the transcriptomic manipulation of the cells composing the blood brain barrier to impact the cellular receptors within the region adjacent to the blood brain barrier. Example drugs or substances may include viral vectors or contrast agents (e.g., substances that my facilitate imaging or intraoperative visualization). In other embodiments, the therapy delivered by IMD 106 is designed to enhance and/or enable cell membrane permeabilization for the purpose of mediating cell transfection by enhancing viral delivery to target cells, enhancing the bioavailability of serologically available pharmaceuticals, enhancing the delivery of tumor-specific marker agents, such as 5-aminolevulinic acid (5-ALA), or for combinatorial efficacy with additional therapy modalities through imparting cellular stress on those cells selectively vulnerable to permeabilization. By being designed to achieve these goals, IMD 106 may be configured (via specific stimulation parameter values) to deliver electrical field therapy that increases blood-brain barrier permeabilization and/or enhances cell membrane permeabilization.

According to the techniques of the disclosure, system 100 may include processing circuitry configured to receive a request to deliver alternating electric field (AEF) therapy, determine therapy parameter values that define the AEF therapy, wherein the AEF therapy comprises delivery of a first electric field and a second electric field, control IMD 106 to deliver the first electric field from a first electrode combination of implanted electrodes, and control IMD 106 to deliver, alternating with the first electric field, the second electric field from a second electrode combination of implanted electrodes different than the first electrode combination. The request may be via user input and/or an automated system request to start AEF therapy delivery.

The electrical fields that IMD 106 alternates over time to produce the AEF therapy may involve different electrode combinations and/or different methods for alternating the electrical fields between different electrode combinations (e.g., different electrodes and/or different polarities of the same or different electrodes). In one example, the first electrode combination includes a first set of electrodes defined as cathodes and a second set of electrodes defined as anodes, and the second electrode combination includes the first set of electrodes defined as anodes and the second set of electrodes defined as cathodes.

In one example in which IMD 106 utilizes 4 different implantable leads (or 4 structures extending from a single lead), the first electrode combination includes a first set of anodes carried by a first lead, the second electrode combination includes a first set of cathodes carried by a second lead different than the first lead, the third electrode combination includes a second set of anodes carried by a third lead different than the first lead and the second lead, and the fourth electrode combination includes a second set of cathodes carried by a fourth lead different than the first lead, the second lead, and the third lead. The first and second electrical fields may generally be orthogonal or oblique to each other. In another example in which two leads are used to deliver AEF therapy, the first electrode combination includes a first set of anodes carried by a first lead, the second electrode combination includes a first set of cathodes carried by a second lead different than the first lead, the third electrode combination comprises a second set of anodes carried by the second lead, and the fourth electrode combination comprises a second set of cathodes carried by the first lead. In some examples, the AEF therapy may include alternating or switching between the first electrode combination and the second electrode combination, where some or all of electrodes of the first lead switch between operating as anodes in the first electrode combination and operating as cathodes in the second electrode combination, and electrodes of the second lead switch between operating as cathodes in the first electrode combination and operating as anodes in the second electrode combination. In other examples, the first and second electrode combinations may utilize completely different electrodes for each anodes and cathodes. In other examples, each electrode combination may utilize one lead for anodes and a different lead for cathodes. The different electrode combinations used to alternate electric fields may share leads or utilize separate leads for each electrode combination. These are only some of the different methods for generating alternating electric fields from an array of implanted electrodes, as other examples are also contemplated. For example, IMD 106 may instead alternate, or sweep through, three or more different electrical fields generated from respective electrode combinations. These larger number of electrical fields may effectively treat a larger number of cells depending on the location of the cells within respect to the location of the implanted electrodes.

Although alternating electric field therapy is generally described as delivering two different electric fields, three or more electric fields may be delivered in other examples. For example, IMD 106 may be configured to deliver three electric fields that are all orthogonal to each other. In other examples, four or more different electric fields may be delivered to the cells in order to affect cells oriented in a variety of different directions. In this manner, IMD 106 may deliver tens or hundreds of different electric fields having different vectors (limited only by the available electrode combinations for delivering the electric fields) by sweeping through a sequence of these electric fields or otherwise delivering these different electric fields in order to affect cells having different orientations. Three electric fields with all different directional vectors may enable three dimensional electrical field treatment of the target tissue.

In some examples, IMD 106 is configured to cycle the AEF therapy on and off according to a predetermined schedule. This predetermined cycle may be set according to the speed of tumor cell division in order to cycle the AEF therapy at a rate that enables the tumor cells are guaranteed to experience a relevant field at least once per cell divisional time to inhibit the division of the cells. In other examples, IMD 106 may be configured to receive temperature data indicative of a temperature of tissue that receives the AEF therapy, determine that the temperature exceeds a threshold temperature, responsive to determining that the temperature exceeds the threshold temperature, terminate delivery of the AEF therapy. This temperature monitoring may reduce the risk of tissue damage due to electrical field induced tissue heating.

IMD 106 may generally use the same pulse or signal frequency for generating the first and second electrical fields of the AEF therapy. In one example, the frequency may be approximately 150 kHz. In another example, the frequency may be approximately 200 kHz. In general, the frequency may be selected from a range of approximately 100 kHz through 300 kHz, but frequencies higher or lower than this range may be used in other examples. In some examples, the frequencies employed by IMD 106 are selected based on the types of cells targeted for treatment. For example, if targeting cancer cells of a certain size (e.g., 13 micrometers in diameter), the IMD 106 delivers therapy with a frequency (e.g., 200 kHz) at which therapy will be more effective for that cell size. In some examples, the frequency or range of frequencies at which the electrical fields are delivered may be selected based on a workup of a patient biopsy or based on a lookup table according to the tumor type and associated distributions of cell sizes. In some examples, the minimum or maximum frequencies may be selected in order to avoid affecting sizes of healthy cells within the electric fields that may differ from the size of the tumor cells.

In some examples, the stimulation frequency at which the maximum force (fₘₐₓ) can be imparted on a spherical particle housed within a dividing cell is inversely related to the relaxation time (τ) possessed by the membrane charging voltage. The relaxation time (τ) can be described by this equation:

$$τ = r {C}_{m} \left(\frac{1}{{σ}_{i}} + \frac{1}{2 {σ}_{e}}\right)$$

The terms within the above formula are as follows: (τ) is the relaxation time of the membrane charging voltage, r is the radius of the cell, Cₘ is the membrane capacitance, and σᵢ and σₑ are the conductivity of the cytoplasm and external medium respectively. Given σᵢ and σₑ are nearly identical, this equation can be simplified to:

$$τ = 1.5 \frac{r {C}_{m}}{{σ}_{i}}$$

Given that within an individual cell the values for r and Cₘ will remain approximately constant within an example frequency range for AEF therapy (100-500 kHz), and again keeping in mind that the frequency at which a maximum force (fₘₐₓ) is imparted on particles within the dividing cell is inversely related to the relaxation time (τ), it can be said that fₘₐₓ is directly related to the cytoplasmic conductivity σᵢ. With that relationship in mind, simulation results are indicative of a relationship between the radius of the cell (r) and fₘₐₓ as follows:

$$f m a x = α \frac{{σ}_{i} d}{r}$$

The terms within this above formula are as follows: fₘₐₓ is the frequency at which maximum force is imparted on a spherical particle within a dividing cell, α represents a constant which was calculated from simulation results within the literature equivalent to 2155 kHz * m * mm/S/nm, σᵢ is the cytoplasmic conductivity, r is the radius of the dividing cell, and d is the membrane thickness represented in nanometers. With this relationship between the frequency for imparting maximal force and cell radius in mind, it can then be determined that with an increase in the cell size, the frequency for affecting the cell will decrease. As in the example of glioblastoma, the standardized cell lines possess an average diameter of approximately 17 mm (or a radius of 8.5 mm), cell membrane thickness is somewhat variable but is reported anywhere between 4 to 10 nm (7 nm for the purposes of this example), and the cytoplasmic conductivity extrapolated from data attained within a melanoma cancer cell, 0.1 S/m. With those values and the value for α mentioned above the resulting optimal frequency to achieve a maximal force on cytoplasmic particles would be 177 kHz (or approximately 200 kHz). This can be compared with a larger cancer cell such as pancreatic cancer which possesses a diameter of 18-22 mm (for the purposes of this example, 20 mm), with all other variables remaining constant, and solving for the equation above the resulting optimal electric field frequency to achieve the maximal force on intracytoplasmic particles would be 151 kHz (or approximately 150 kHz). Both these frequencies are shown to be effective, suggesting validity to the relationship between the variables highlighted above. Therefore, if a particular cell type can be selectively impacted by a specific TTF therapy (or AEF therapy) frequency, it is feasible for a system to selectively avoid the impact on normal cell types that possess differing values of cell radius or cytoplasmic conductivity.

Given that AEF therapy has been demonstrated to impart an increase in cell volume within those cancer cells experiencing the therapy, in part due to the enhanced proportion of cells that occupy the G₀/G₁ phase of the cell cycle, and that the optimal frequency for maximal efficacy of AEF therapy if cell size dependent (as above), the system may deliver a sweep of different frequencies such as an interleaved or continuously sweeping protocol ranging between 100 kHz to 250 kHz would provide an optimal treatment for the diverse cell population of the tumor

In some examples, system 100 may be configured to determine, or recommend for user approval, one or more stimulation parameters that at least partially define the AEF therapy. For example, programmer 104 may include a user interface configured to receive user input indicative of target tissue to receive AEF therapy. Programmer 104 may be configured to determine, based on the user input, the first electrode combination and the second electrode combination. In this manner, system 100 can achieve therapy of desired tissue, such as a glioblastoma tumor or other tissue of concern. Alternatively, or in addition, programmer 104 may include a user interface configured to receive user input indicative of tissue to avoid receiving AEF therapy. Since programmer 104 may be a patient or clinician programmer, the user interface may be configured to receive input from a clinician or a patient. However, in some examples, the user interface may provide additional options or expanded customizability for clinicians when compared to patients. In some embodiments, the IMD 106 is configured to determine, e.g., using signals sensed by the electrodes, that electric fields are reaching a particular tissue structure. In some examples, one or more of the electrodes may be located in or near a non-target tissue to indicate the presence of electric fields at the non-target tissue (e.g., a specific recording electrode(s)). The IMD 106 (either alone or in combination with external devices) can adjust the applied therapy to reduce or eliminate the applied electric fields (or the effects of the applied electric fields) at that particular tissue structure. Programmer 104 may then determine, based on the user input, the first electrode combination and the second electrode combination. System 100 can then attempt to reduce the effect of AEF therapy on non-target tissues. In some examples, system 100 may receive user input indicative of target tissue and/or tissue to avoid from a remote device over a network to support remote programmer options for system 100.

System 100 may also determine stimulation parameters based on feedback regarding the state of patient 112 and/or tissue of the patient. For example, programmer 104 and/or IMD 106 may adjust one or more stimulation parameters that at least partially defines the AEF therapy based on histological data obtained from a sample of tissue affected by the AEF therapy. In another example, programmer 104 and/or IMD 106 may determine target tissue for AEF therapy based on water content data obtained from magnetic resonance imaging (MRI) data, and determine, based on the target tissue, the first electrode combination and the second electrode combination for delivery of the AEF therapy. In some examples, determining the electrode combinations may include determining the location, e.g., based on predictive computational models of electric field intensity in tissue, at which one or more leads should be located in order to deliver AEF therapy to the target tissue. As another example, programmer 104 and/or IMD 106 may determine target tissue for AEF therapy based on impedance tomography data obtained from sensed electrical potentials sensed from two or more of the implanted electrodes (and/or external electrodes disposed to record electric fields), and determine, based on the target tissue, at least the first electrode combination and the second electrode combination to deliver the AEF therapy. System 100 may also map AEF features to anatomy to inform AEF therapy planning and/or adjustments over time. For example, programmer 104 may be configured to generate an AEF dosimetry metric for anatomy that receives the AEF therapy and map the AEF dosimetry across target tissue of the anatomy. This AEF dosimetry map may inform which tissues within the anatomy are receiving different strengths of the electrical fields. Programmer 104 may also display the map of the AEF dosimetry with respect to the anatomy.

IMD 106 may alternate the electrical fields in AEF therapy by delivering the electrical fields from different electrodes and/or electrodes with different polarities. In one example, IMD 106 may continually shift the polarities of the electrodes in one direction with respect to the electrode array. The first electrode combination may include a first set of electrodes defined as cathodes and a second set of electrodes defined as anodes, the second electrode combination may include a third set of electrodes defined as anodes and a fourth set of electrodes defined as cathodes, where the third set of electrodes are adjacent to the first set of electrodes in one direction on a first lead, and the fourth set of electrodes are adjacent to the second set of electrodes in the one direction on a second lead. In some examples, electrode combinations adjacent each other may be 180 degrees out of phase with each other in order to provide a maximum amount of change in voltage between the tissue separating the adjacent electrode contacts. In some examples, to accomplish the enhancement of AEF therapy, the electrode near or within the non-target tissue could be paired to the local stimulating electrodes in a 180° or π radians phase shifted configuration along the stimulation sinusoidal waveform. In doing so, the resulting electric field magnitude experienced by the non-target tissue may be higher due to the larger peak-to-peak differential in voltage between the two electrodes. To accomplish a reduction or elimination of AEF therapy within the non-target tissue region, the system may implement a 0° or 0π radians phase shifting configuration between the local electrode and the remote stimulating electrode. By conducting the stimulation in this manner, there is less permissible of differential established in the peak-to-peak voltage experienced by the local tissue and therefore a reduction in the resulting electric field.

In another example, the electrode combinations may be selected from a cube configuration where the selectable electrodes for each electrode combination form the eight vertices of a cube. In this example, the first electrode combination includes a first set of electrodes defined as cathodes and a second set of electrodes defined as anodes in a first paired configuration from the cube configuration, and the second electrode combination includes the first set of electrodes defined as anodes and the second set of electrodes defined as cathodes in a second paired configuration from the cube configuration.

As shown in FIG. 1, the electrodes (e.g., at least two electrodes) used to deliver the AEF therapy are carried by an electrode array positioned adjacent a resection bed of tissue (e.g., a tissue resection region). In some examples, system 100 may generate the electrical field modulation, such as AEF, using one or more electrodes implanted within the skull, outside of the skull and under the skin (e.g., subcutaneous), and/or external to the skin of patient 122.

Generally, AEF therapy is described herein as a treatment to already present tumors, such as glioblastomas. In other examples, the application of AEF therapy can reduce the extent of metastatic tumor burden and seeding of tumors from a remote tumor source. Therefore, AEF treatment could be delivered to protect tissue regions from metastatic spread. For example, AEF could be utilized to provide global brain protection in the setting of a known malignant tumor within the body, particularly those that have a propensity for cerebral dissemination (e.g., Melanoma). AEF could be delivered to prevent additional metastatic spread of tumor within the organ system of current metastatic dissemination. In addition, AEF implant planning could be provided for the protection of certain neurological function (e.g., motor function), such that the implant system 100 would be focused on treatment to the pre-central gyrus and/or corticospinal tract to preserve its function and avoid seeding.

The architecture of system 100 illustrated in FIG. 1 is shown as an example. The techniques as set forth in this disclosure may be implemented in the example system 100 of FIG. 1, as well as other types of systems not described specifically herein. Nothing in this disclosure should be construed so as to limit the techniques of this disclosure to the example architecture illustrated by FIG. 1.

System 100 is generally described as including IMD 106 and external programmer 104. However, in other examples, an external medical device may be configured to perform any of the techniques described herein or described with respect to IMD 106. The external medical device may be coupled to percutaneous leads or other devices that pass through the skin in order to dispose implanted electrodes at various locations within patient for at least partially delivering electric field therapy and/or sensing signals as described herein. Additionally, or alternatively, the external device may be coupled to external electrodes configured to at least partially deliver electric field therapy and/or sense signals as described herein. The external medical device may be configured to communicate with programmer 104 and/or partially or fully incorporate structures to perform the various functionality described with respect to programmer 104.

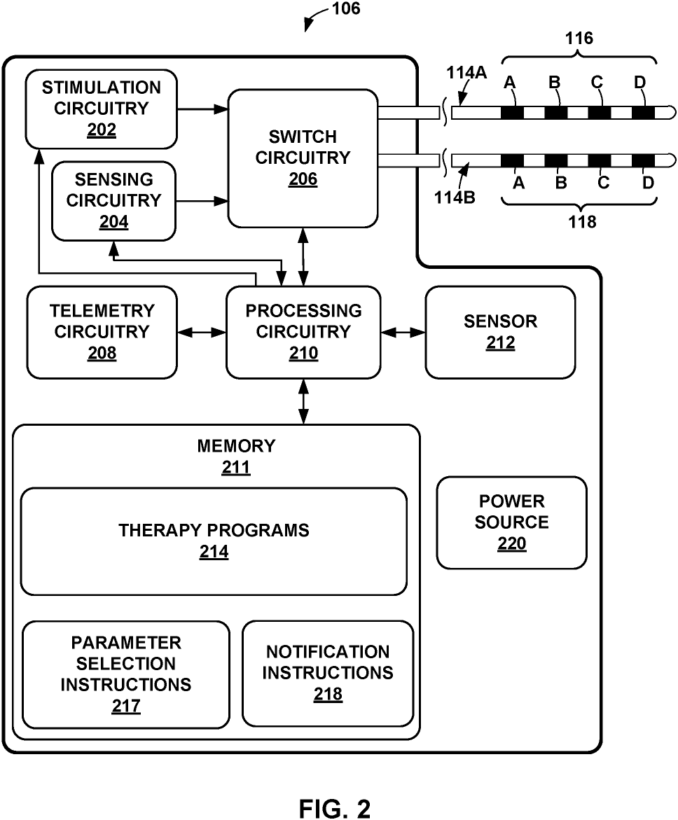

FIG. 2 is a block diagram of the example IMD 106 of FIG. 1 for delivering AEF therapy. In the example shown in FIG. 2, IMD 106 includes processing circuitry 210, memory 211, stimulation generator 202, sensing module 204, switch module 206, telemetry module 208, sensor 212, and power source 220. Each of these modules may be or include electrical circuitry configured to perform the functions attributed to each respective module. For example, processing circuitry 210 may include processing circuitry, switch module 206 may include switch circuitry, sensing module 204 may include sensing circuitry, and telemetry module 208 may include telemetry circuitry. Switch circuitry 206 may not be used for multiple current source and sink configurations, but one or more switches may still be used to disconnect sensing module 204 from the source and sinks in such a configuration. Although switch circuitry 206 is shown in the housing of FIG. 106, switch circuitry 206 or another switch circuit (e.g., a multiplexer) may be located external of IMD 106 in other example. Memory 211 may include any volatile or non-volatile media, such as a random-access memory (RAM), read only memory (ROM), non-volatile RAM (NVRAM), electrically erasable programmable ROM (EEPROM), flash memory, and the like. Memory 211 may store computer-readable instructions that, when executed by processing circuitry 210, cause IMD 106 to perform various functions. Memory 211 may be a storage device or other non-transitory medium.

In the example shown in FIG. 2, memory 211 stores therapy programs 214 that include respective stimulation parameter sets that define AEF therapy. Each stored therapy program 214 defines a particular set of electrical stimulation parameters (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated.

Memory 211 may also include parameter selection instructions 217 and notification instructions 218. Parameter selection instructions 217 may include instructions that control processing circuitry 210 selecting different stimulation parameter values such as electrode combinations, amplitudes, pulse frequencies, or other parameter values for compensating for various locations of target tissue or feedback related to changes in patient condition or tissue state. Parameter selection instructions 217 may include instructions for processing circuitry 210 to select parameter values based on various feedback variables. Notification instructions 218 may define instructions that control processing circuitry 210 actions such as transmitting an alert or other notification to an external device, such as programmer 104, that therapy is on or off, or if changes to AEF therapy have been made or are recommended.

In some examples, the sense and stimulation electrode combinations may include the same subset of electrodes 116, 118, a housing of IMD 106 functioning as an electrode, or may include different subsets or combinations of such electrodes. Thus, memory 211 can store a plurality of sense electrode combinations and, for each sense electrode combination, store information identifying the stimulation electrode combination that is associated with the respective sense electrode combination. The associations between sense and stimulation electrode combinations can be determined, e.g., by a clinician or automatically by processing circuitry 210. In some examples, corresponding sense and stimulation electrode combinations may comprise some or all of the same electrodes. In other examples, however, some or all of the electrodes in corresponding sense and stimulation electrode combinations may be different. For example, a stimulation electrode combination may include more electrodes than the corresponding sense electrode combination in order to increase the efficacy of the AEF therapy.

Stimulation generator 202, under the control of processing circuitry 210, generates stimulation signals for delivery to patient 112 via selected combinations of electrodes 116, 118. An example range of electrical stimulation parameters believed to be effective in AEF therapy to manage cellular activity include:
1. Frequency (e.g., waveform frequency or pulse rate): between approximately 50 kHz and approximately 500 kHz, such as between approximately 100 kHz to 300 kHz, or such as approximately 150 kHz or 200 kHz.
2. In the case of a voltage controlled system, Voltage Amplitude: between approximately 0.1 volts and approximately 50 volts, such as between approximately 2 volts and approximately 10 volts.
3. In the alternative case of a current controlled system, Current Amplitude: between approximately 0.2 milliamps to approximately 100 milliamps, such as between approximately 1.3 milliamps and approximately 2.0 milliamps.
4. Pulse Width: between approximately 1 microseconds and approximately 10 microseconds, such as between approximately 1 microseconds and approximately 5 microseconds, or between approximately 2 microseconds and approximately 10 microseconds.
5. Cycle time (e.g., communication time), which is the time a waveform remains consistent before switching off or switching to a new waveform. The cycle time may be selected from a range of 30 seconds and 30 minutes, or within a range from 1 minute to 10 minutes. Shorter and longer cycle times may be used in other examples.

Accordingly, in some examples, stimulation generator 202 generates electrical stimulation signals in accordance with the electrical stimulation parameters noted above. Other ranges of therapy parameter values may also be useful, and may depend on the target stimulation site within patient 112. While stimulation pulses are described, stimulation signals may be of any form, such as continuous-time signals (e.g., sine waves) or the like. Stimulation signals configured to elicit ECAPs or other evoked physiological signals may be similar or different from the above parameter value ranges. In addition, sensing circuitry 204 may be configured to sense signals via one or more electrode combinations on one or more leads 114 (e.g., the same or different electrodes may deliver stimulation and sense electrical signals).

Processing circuitry 210 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 may control stimulation generator 202 according to therapy programs 214 stored in memory 211 to apply particular stimulation parameter values specified by one or more of programs, such as voltage amplitude or current amplitude, pulse width, or pulse rate.

In the example shown in FIG. 2, the set of electrodes 116 includes electrodes 116A, 116B, 116C, and 116D, and the set of electrodes 118 includes electrodes 118A, 118B, 118C, and 118D. Processing circuitry 210 also controls switch module 206 to apply the stimulation signals generated by stimulation generator 202 to selected combinations of electrodes 116, 118. In particular, switch module 204 may couple stimulation signals to selected conductors within leads 114, which, in turn, deliver the stimulation signals across selected electrodes 116, 118. Switch module 206 may be a switch array, switch matrix, multiplexer, or any other type of switching module configured to selectively couple stimulation energy to selected electrodes 116, 118 and to selectively sense neurological brain signals with selected electrodes 116, 118. Hence, stimulation generator 202 is coupled to electrodes 116, 118 via switch module 206 and conductors within leads 114. In some examples, however, IMD 106 does not include switch module 206, such as if each electrode is assigned a respective current and sink (e.g., independent current source).

Stimulation generator 202 may be a single channel or multi-channel stimulation generator. In particular, stimulation generator 202 may be capable of delivering a single stimulation pulse, multiple stimulation pulses, or a continuous signal at a given time via a single electrode combination or multiple stimulation pulses at a given time via multiple electrode combinations. In some examples, however, stimulation generator 202 and switch module 206 may be configured to deliver multiple channels on a time-interleaved basis. For example, switch module 206 may serve to time divide the output of stimulation generator 202 across different electrode combinations at different times to deliver multiple programs or channels of stimulation energy to patient 112 (e.g., cycling between regimes of stimulation on a fixed or variable sequence). Alternatively, stimulation generator 202 may comprise multiple voltage or current sources and sinks that are coupled to respective electrodes to drive the electrodes as cathodes or anodes. In this example, IMD 106 may not require the functionality of switch module 206 for time-interleaved multiplexing of stimulation via different electrodes.

Electrodes 116, 118 on respective leads 114 may be constructed of a variety of different designs. For example, one or both of leads 114 may include two or more electrodes at each longitudinal location along the length of the lead, such as multiple electrodes at different perimeter locations around the perimeter of the lead at each of the locations A, B, C, and D. On one example, the electrodes may be electrically coupled to switch module 206 via respective wires that are straight or coiled within the housing or the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 114. These and other constructions may be used to create a lead with a complex electrode geometry.

Although sensing module 204 is incorporated into a common housing with stimulation generator 202 and processing circuitry 210 in FIG. 2, in other examples, sensing module 204 may be in a separate housing from IMD 106 and may communicate with processing circuitry 210 via wired or wireless communication techniques. Example neurological brain signals include, but are not limited to, a signal generated from local field potentials (LFPs) within one or more regions of brain 28. EEG and ECoG signals are examples of other types of electrical signals that may be measured within brain 120 and/or outside of brain 120. Other examples include sensed signals representative of electric field or voltage gradients caused by a remote electrode as recorded by a proximal electrode or electrode pair. Instead of, or in addition to, LFPs, IMD 106 may be configured to detect patterns of single-unit activity and/or multi-unit activity. IMD 106 may sample this activity at rates above 1,000 Hz, and in some examples within a frequency range of 6,000 Hz to 500,000 Hz. IMD 106 may identify the wave-shape of single units and/or an envelope of unit modulation that may be features used to differentiate or rank electrodes. In some examples, this technique may include phase-amplitude coupling to the envelope or to specific frequency bands in the LFPs sensed from the same or different electrodes. In some examples, the sampling technique may be set to identify the electric field strength at any location. For example, IMD 106 may include a peak following circuitry that holds the amplitude of a field of a specific frequency for later sampling. Alternatively, the response of a resonant circuit may be tuned to the AEF frequency might sampled to infer the field strength of the desired signal. In some examples, IMD 106 may be configured to detect a geometric response within the network of 114 electrodes in response to a single-pulse electrical stimulation generated within the system. The utilization of a basis profile curve algorithm to analyze this geometric response as sensed within the multitude of 114 electrodes within the system can permit diagnostics, such as demonstration of patters indicative of depression, anxiety, or tumor progression within the cerebral environment. IMD 106 may conduct this real-time diagnostic modality in an interleaved manner to permit ongoing stimulation with periodic analysis.

Sensor 212 may include one or more sensing elements that sense values of a respective patient parameter, such as patient activity (e.g., movement and/or sleep). For example, sensor 212 may include one or more accelerometers, optical sensors, chemical sensors, temperature sensors, pressure sensors, or any other types of sensors. Sensor 212 may output patient parameter values that may be used as feedback to control delivery of AEF therapy. IMD 106 may include additional sensors within the housing of IMD 106 and/or coupled via one of leads 114 or other leads. In addition, IMD 106 may receive sensor signals wirelessly from remote sensors via telemetry module 208, for example. In some examples, one or more of these remote sensors may be external to patient (e.g., carried on the external surface of the skin, attached to clothing, or otherwise positioned external to the patient).

Telemetry module 208 supports wireless communication between IMD 106 and an external programmer 104 or another computing device under the control of processing circuitry 210. Processing circuitry 210 of IMD 106 may receive, as updates to programs, values for various stimulation parameters such as magnitude and electrode combination, from programmer 104 via telemetry module 208. The updates to the therapy programs may be stored within therapy programs 214 portion of memory 211. In addition, processing circuitry 210 may control telemetry module 208 to transmit alerts or other information to programmer 104 that indicate a lead moved with respect to tissue. Telemetry module 208 in IMD 106, as well as telemetry modules in other devices and systems described herein, such as programmer 104, may accomplish communication by radiofrequency (RF) communication techniques. In addition, telemetry module 208 may communicate with external medical device programmer 104 via proximal inductive interaction of IMD 106 with programmer 104. Accordingly, telemetry module 208 may send information to external programmer 104 on a continuous basis, at periodic intervals, or upon request from IMD 106 or programmer 104.

Power source 220 delivers operating power to various components of IMD 106. Power source 220 may include a small rechargeable or non-rechargeable battery and a power generation circuit to produce the operating power. Recharging may be accomplished through proximal inductive interaction between an external charger and an inductive charging coil within IMD 220. In some examples, power requirements may be small enough to allow IMD 220 to utilize patient motion and implement a kinetic energy-scavenging device to trickle charge a rechargeable battery. In other examples, traditional batteries may be used for a limited period of time. In other examples, IMD 106 may include a power receiving antenna an corresponding circuitry to continually receive external power that enables IMD 106 to deliver electric field therapy indefinitely without possible internal power source drain.

According to the techniques of the disclosure, processing circuitry 210 of IMD 106 delivers, electrodes 116, 118 interposed along leads 114 (and optionally switch module 206), electrical stimulation therapy to patient 112. The AEF therapy is defined by one or more therapy programs 214 having one or more parameters stored within memory 211. For example, the one or more parameters include a current amplitude (for a current-controlled system) or a voltage amplitude (for a voltage-controlled system), a pulse rate or frequency, and a pulse width, or quantity of pulses per cycle. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time.

In some examples, the plurality of electrode combinations includes at least one electrode combination comprising electrodes disposed at different positions around a perimeter, or circumference, of the longitudinal axis lead. In some examples, at least one electrode combination includes electrodes disposed at different positions along a longitudinal axis of the lead implanted in the patient. These electrodes may be placed at the same or different radial positions with respect to the longitudinal axis.

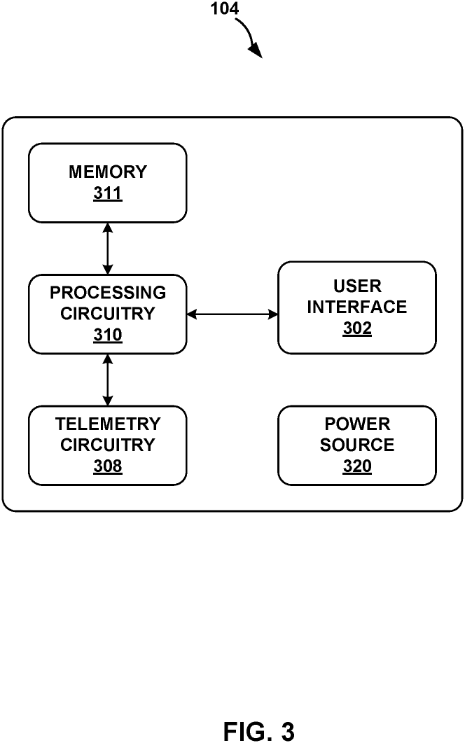

FIG. 3 is a block diagram of the external programmer 104 of FIG. 1 for planning and/or controlling delivery of AEF therapy using available electrodes according to an example of the techniques of the disclosure. Although programmer 104 may generally be described as a hand-held device, programmer 104 may be a larger portable device or a more stationary device. In some examples, programmer 104 may be referred to as a tablet computing device or a smart phone computing device. In addition, in other examples, programmer 104 may be included as part of a bed-side monitor, an external charging device or include the functionality of an external charging device. As illustrated in FIG. 3, programmer 104 may include a processing circuitry 310, memory 311, user interface 302, telemetry module 308, and power source 320. Memory 311 may store instructions that, when executed by processing circuitry 310, cause processing circuitry 310 and external programmer 104 to provide the functionality ascribed to external programmer 104 throughout this disclosure. Each of these components, or modules, may include electrical circuitry that is configured to perform some or all of the functionality described herein. For example, processing circuitry 310 may include processing circuitry configured to perform the processes discussed with respect to processing circuitry 310.

In general, programmer 104 comprises any suitable arrangement of hardware, alone or in combination with software and/or firmware, to perform the techniques attributed to programmer 104, and processing circuitry 310, user interface 302, and telemetry module 308 of programmer 104. In various examples, programmer 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. Programmer 104 also, in various examples, may include a memory 311, such as RAM, ROM, PROM, EPROM, EEPROM, flash memory, a hard disk, a CD-ROM, comprising executable instructions for causing the one or more processors to perform the actions attributed to them. Moreover, although processing circuitry 310 and telemetry module 308 are described as separate modules, in some examples, processing circuitry 310 and telemetry module 308 may be functionally integrated with one another. In some examples, processing circuitry 310 and telemetry module 308 correspond to individual hardware units, such as ASICs, DSPs, FPGAs, or other hardware units.

Memory 311 (e.g., a storage device) may store instructions that, when executed by processing circuitry 310, cause processing circuitry 310 and programmer 104 to provide the functionality ascribed to programmer 104 throughout this disclosure. For example, memory 311 may include instructions that cause processing circuitry 310 to obtain a parameter set from memory, present a model of patient anatomy for predicting electrical field strengths, provide an interface that recommends or otherwise facilitates parameter value selection, or receive a user input and send a corresponding command to IMD 106, or instructions for any other functionality. In addition, memory 311 may include a plurality of programs, where each program includes a parameter set that defines stimulation therapy.

User interface 302 may include a button or keypad, lights, a speaker for voice commands, a display, such as a liquid crystal (LCD), light-emitting diode (LED), or organic light-emitting diode (OLED). In some examples the display may be a presence-sensitive screen, such as a touch screen. User interface 302 may be configured to display any information related to the delivery of stimulation therapy, detected trigger events, progression of therapy, suggested stimulation parameter values, sensed patient parameter values, or any other such information. User interface 302 may also receive user input via user interface 302. The input may be, for example, in the form of pressing a button on a keypad or selecting an icon from a touch screen.

Telemetry module 308 may support wireless communication between IMD 106 and programmer 104 under the control of processing circuitry 310. Telemetry module 308 may also be configured to communicate with another computing device via wireless communication techniques, or direct communication through a wired connection. In some examples, telemetry module 308 provides wireless communication via an RF or proximal inductive medium. In some examples, telemetry module 308 includes an antenna, which may take on a variety of forms, such as an internal or external antenna. In some examples, IMD 106 and/or programmer 104 may communicate with remote servers via one or more cloud-services in order to deliver and/or receive information between a clinic and/or programmer.

Examples of local wireless communication techniques that may be employed to facilitate communication between programmer 104 and IMD 106 include RF communication according to the 802.11 or Bluetooth specification sets or other standard or proprietary telemetry protocols. Security protocols and encryption techniques may be applied to enhance the security of the communication techniques. In addition, other external devices may be capable of communicating with programmer 104 without needing to establish a secure wireless connection. As described herein, telemetry module 308 may be configured to transmit a spatial electrode movement pattern or other stimulation parameter values to IMD 106 for delivery of stimulation therapy.

FIG. 4 is a block diagram illustrating an example system 124 that includes an external device, such as a server 130, and one or more computing devices 132A-132N, that are coupled to IMD 106 and external programmer 104 shown in FIG. 1 via a network 126. In this example, IMD 106 may use its telemetry circuit to communicate with external programmer 104 via a first wireless connection, and to communication with an access point 128 via a second wireless connection.

In the example of FIG. 4, access point 128, external programmer 104, server 130, and computing devices 132A-132N are interconnected, and able to communicate with each other, through network 126. In some cases, one or more of access point 128, external programmer 104, server 130, and computing devices 132A-132N may be coupled to network 126 through one or more wireless connections. IMD 106, external programmer 104, server 130, and computing devices 132A-132N may each comprise one or more processors, such as one or more microprocessors, digital signal processors (DSPs), application specific integrated circuits (ASICs), field programmable gate arrays (FPGAs), programmable logic circuitry, or the like, that may perform various functions and operations, such as those described in this disclosure.

Access point 128 may comprise a device, such as a home monitoring device, that connects to network 126 via any of a variety of connections, such as telephone dial-up, digital subscriber line (DSL), or cable modem connections. In other embodiments, access point 128 may be coupled to network 126 through different forms of connections, including wired or wireless connections.

During operation, IMD 106 may collect and store various forms of data. For example, IMD 106 may collect sensed posture state information during therapy that indicate how patient 112 moves throughout each day. IMD 106 may store usage statistics (e.g., delivery times in hours per day, percentage of on time, compliance to the dosing schedule, etc.) for later presentation to a user or otherwise evaluating therapy and/or patient compliance. In some cases, IMD 106 may directly analyze the collected data to evaluate the status of the patient and the delivery of AEF therapy or any other aspects of the patient. In other cases, however, IMD 106 may send stored data relating to AEF therapy to external programmer 104 and/or server 130, either wirelessly or via access point 128 and network 126, for remote processing and analysis.

For example, IMD 106 may sense, process, trend and evaluate sensed data and/or AEF therapy information. This communication may occur in real time, and network 126 may allow a remote clinician to review the data representative of AEF therapy by receiving a presentation of the data on a remote display, e.g., computing device 132A. Alternatively, processing, trending and evaluation functions may be distributed to other devices such as external programmer 104 or server 130, which are coupled to network 126. In addition, AEF therapy data may be archived by any of such devices, e.g., for later retrieval and analysis by a clinician.

In some cases, server 130 may be configured to provide a secure storage site for archival of AEF therapy information that has been collected from IMD 106 and/or external programmer 104. Network 126 may comprise a local area network, wide area network, or global network, such as the Internet. In other cases, external programmer 104 or server 130 may assemble AEF therapy information in web pages or other documents for viewing by trained professionals, such as clinicians, via viewing terminals associated with computing devices 132A-132N. System 124 may be implemented, in some aspects, with general network technology and functionality similar to that provided by the Medtronic CareLink® Network developed by Medtronic, Inc., of Minneapolis, MN.

Although some examples of the disclosure may involve AEF therapy information and data, system 124 may be employed to distribute any information relating to the treatment of patient 112 and the operation of any device associated therewith. For example, system 124 may allow therapy errors or device errors to be immediately reported to the clinician. In addition, system 124 may allow the clinician to remotely intervene in the therapy and reprogram IMD 106, patient programmer 104, or communicate with patient 112. In an additional example, the clinician may utilize system 124 to monitor multiple patients and share data with other clinicians in an effort to coordinate rapid evolution of effective treatment of patients..

FIGS. 5A and 5B are conceptual diagrams of example leads 400 and 410, respectively, with respective electrodes carried by the lead. As shown in FIGS. 5A and 5B, leads 400 and 410 are embodiments of leads 114 shown in FIG. 1. As shown in FIG. 5A, lead 400 includes four electrode levels 404 (includes levels 404A-404D) mounted at various lengths of lead housing 402. Lead 400 is inserted into through cranium 122 to a target position within brain 18. In some examples, external electrodes may be used instead of, or in addition to, leads such as lead 400.

Lead 400 is implanted within brain 120 at a location determined by the clinician that may be near an anatomical region to receive AEF therapy, such as a tumor location or resection bed. Electrode levels 404A, 404B, 404C, and 404D are equally spaced along the axial length of lead housing 402 at different axial positions. Each electrode level 404 may have one, two, three, or more electrodes located at different angular positions around the circumference (e.g., around the perimeter) of lead housing 402. As shown in FIG. 5A, electrode level 404A and 404D include a single respective ring electrode, and electrode levels 404B and 404C each include three electrodes at different circumferential positions. This electrode pattern may be referred to as a 1-3-3-1 lead in reference to the number of electrodes from the proximal end to the distal end of lead 400. Electrodes of one circumferential location may be lined up on an axis parallel to the longitudinal axis of lead 400. Alternatively, electrodes of different electrode levels may be staggered around the circumference of lead housing 402. In addition, lead 400 or 410 may include asymmetrical electrode locations around the circumference, or perimeter, of each lead or electrodes of the same level that have different sizes. These electrodes may include semi-circular electrodes that may or may not be circumferentially aligned between electrode levels.

Lead housing 402 may include a radiopaque stripe or other one or more radiopaque marker (not shown) along the outside of the lead housing. The radiopaque stripe corresponds to a certain circumferential location that allows lead 400 to be imaged and reliably localized when implanted in patient 112. Using the images of patient 112, the clinician can use the radiopaque stripe as a marker for the exact orientation of lead 400 within the brain of patient 112. Orientation of lead 400 may be needed to easily program the stimulation parameters by generating the correct electrode configuration to match the stimulation field defined by the clinician. In other embodiments, a marking mechanism other than a radiopaque stripe may be used to identify the orientation of lead 400. These marking mechanisms may include something similar to a tab, detent, or other structure on the outside of lead housing 402. In some embodiments, the clinician may note the position of markings along a lead wire during implantation to determine the orientation of lead 400 within patient 112. In some examples, programmer 104 may update the orientation of lead 400 in visualizations based on the movement of lead 400 from sensed signals. Any mechanical or radiopaque markers may be provided in any leads and/or lead structures described herein in order to identify locations of the leads and/or electrodes implanted within the patient and relative to target tissue.

FIG. 5B illustrates lead 410 that includes multiple electrodes at different respective circumferential positions at each of levels 414A-414D. Similar to lead 400, lead 410 is inserted through a burr hole, craniostomy, or craniotomy in cranium 122 to a target location within brain 120. Lead 410 includes lead housing 412. Four electrode levels 414 (414A-414D) are located at the distal end of lead 410. Each electrode level 414 is evenly spaced from the adjacent electrode level and includes two or more electrodes. In one embodiment, each electrode level 414 includes three, four, or more electrodes distributed around the circumference of lead housing 412. Therefore, lead 410 includes 414 electrodes in a preferred embodiment. Each electrode may be substantially rectangular in shape. Alternatively, the individual electrodes may have alternative shapes, e.g., circular, oval, triangular, rounded rectangles, or the like.

In alternative embodiments, electrode levels 404 or 414 are not evenly spaced along the longitudinal axis of the respective leads 400 and 410. For example, electrode levels 404C and 404D may be spaced approximately 3 millimeters (mm) apart while electrodes 404A and 404B are 10 mm apart. Variable spaced electrode levels may be useful in reaching target anatomical regions deep within brain 120 while avoiding potentially undesirable anatomical regions. The variable spacing may also be utilized to enhance the resulting AEF therapy generated between a pair of electrodes carried on any of leads 400 or 410. Further, the electrodes in adjacent levels need not be aligned in the direction as the longitudinal axis of the lead, and instead may be oriented diagonally with respect to the longitudinal axis.

Leads 400 and 410 are substantially rigid to prevent the implanted lead from varying from the expected lead shape. Leads 400 or 410 may be substantially cylindrical in shape. In other embodiments, leads 400 or 410 may be shaped differently than a cylinder. For example, the leads may include one or more curves to reach target anatomical regions of brain 120. In some embodiments, leads 400 or 410 may be similar to a flat paddle lead or a conformable lead shaped for patient 112. Also, in other embodiments, leads 400 and 410 may any of a variety of different polygonal cross sections (e.g., triangle, square, rectangle, octagonal, etc.) taken transverse to the longitudinal axis of the lead.

As shown in the example of lead 400, the plurality of electrodes of lead 400 includes a first set of three electrodes disposed at different respective positions around the longitudinal axis of the lead and at a first longitudinal position along the lead (e.g., electrode level 404B), a second set of three electrodes disposed at a second longitudinal position along the lead different than the first longitudinal position (e.g., electrode level 404C), and at least one ring electrode disposed at a third longitudinal position along the lead different than the first longitudinal position and the second longitudinal position (e.g., electrode level 404A and/or electrode level 404D). In some examples, electrode level 404D may be a bullet tip or cone shaped electrode that covers the distal end of lead 402.

FIGS. 5C-5F are transverse cross-sections of example stimulation leads having one or more electrodes around the circumference of the lead. As shown in FIGS. 5C-5F, one electrode level, such as one of electrode levels 404 and 414 of leads 400 and 410, are illustrated to show electrode placement around the perimeter, or around the longitudinal axis, of the lead. FIG. 5C shows electrode level 500 that includes circumferential electrode 502. Circumferential electrode 502 encircles the entire circumference of electrode level 500 and may be referred to as a ring electrode in some examples. Circumferential electrode 502 may be utilized as a cathode or anode as configured by the user interface. Any of the electrodes of FIGS. 5A-5F may be configured to act as a sensing electrode, or as part of a sensing electrode combination, within a tissue environment.

FIG. 5D shows electrode level 510 which includes two electrodes 512 and 514. Each electrode 512 and 514 wraps approximately 170 degrees around the circumference of electrode level 510. Spaces of approximately 10 degrees are located between electrodes 512 and 514 to prevent inadvertent coupling of electrical current between the electrodes. Smaller or larger spaces between electrodes (e.g., between 10 degrees and 30 degrees) may be provided in other examples. Each electrode 512 and 514 may be programmed to act as an anode or cathode.

FIG. 5E shows electrode level 520 which includes three equally sized electrodes 522, 524 and 526. Each electrode 522, 524 and 526 encompass approximately 110 degrees of the circumference of electrode level 520. Similar to electrode level 510, spaces of approximately 10 degrees separate electrodes 522, 524 and 526. Smaller or larger spaces between electrodes (e.g., between 10 degrees and 30 degrees) may be provided in other examples. Electrodes 522, 524 and 526 may be independently programmed as an anode or cathode for stimulation.

FIG. 5F shows electrode level 530 which includes four electrodes 532, 534, 536 and 538. Each electrode 532, 534, 536 and 538 covers approximately 80 degrees of the circumference with approximately 10 degrees of insulation space between adjacent electrodes. Smaller or larger spaces between electrodes (e.g., between 10 degrees and 30 degrees) may be provided in other examples. In other embodiments, up to ten or more electrodes may be included within an electrode level. In alternative embodiments, consecutive electrode levels of lead 114 may include a variety of electrode levels 500, 510, 520, and 530. For example, lead 114 (or any other lead described herein) may include electrode levels that alternate between electrode levels 510 and 530 depicted in FIGS. 5D and 5F. In this manner, various stimulation field shapes may be produced within brain 120 of patient 112. Further the above-described sizes of electrodes within an electrode level are merely examples, and the invention is not limited to the example electrode sizes.

Also, the insulation space, or non-electrode surface area, may be of any size. Generally, the insulation space is between approximately 1 degree and approximately 20 degrees. More specifically, the insulation space may be between approximately 5 and approximately 15 degrees. In other examples, insulation space may be between approximately 10 degrees and 30 degrees or larger. Smaller insulation spaces may allow a greater volume of tissue to be stimulated. In alternative embodiments, electrode size may be varied around the circumference of an electrode level. In addition, insulation spaces may vary in size as well. Such asymmetrical electrode levels may be used in leads implanted at tissues needing certain shaped stimulation fields. In some examples, the insulation region of the lead may include a projection that extends radially outward from the lead body. Although not shown, any lead or electrode array may include one or more fixation elements (e.g., tines, screws, electrode shapes, adhesives, etc.) that enable the lead or electrodes to be relatively fixed in position with respect to surrounding tissue.

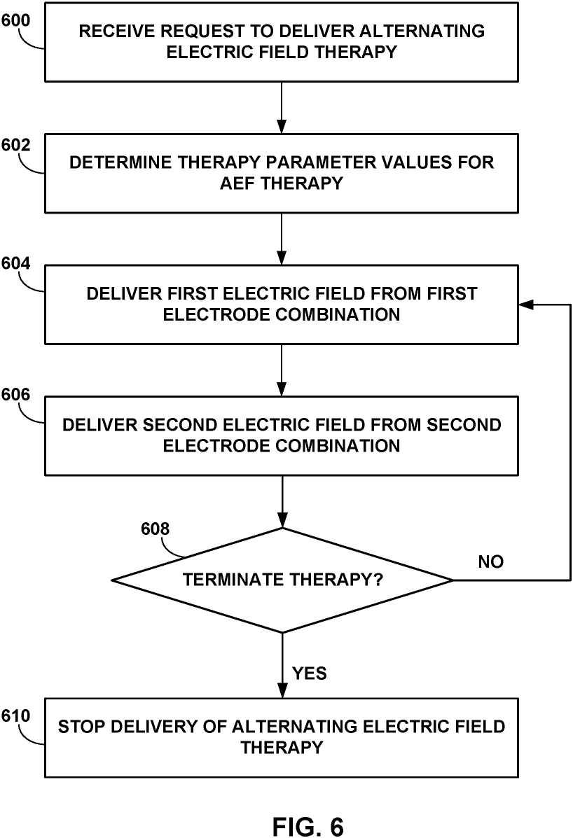

FIG. 6 is a flowchart illustrating an example technique for delivering AEF therapy to a patient. The technique of FIG. 6 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 6 in other examples. The technique of FIG. 6 may apply to therapies other than AEF therapy in a similar manner.

As shown in the example of FIG. 6, processing circuitry 210 receives a request to deliver alternating electric field (AEF) therapy (600), such as from external programmer 104 or a pre-programmed delivery schedule. Processing circuitry 210 then determines therapy parameter values for AEF therapy (602). This determination may be retrieval of parameter values from memory or determining one or more parameter values based on a delivery schedule, sensed data, or any other information. For example, the parameter values may be based on the spatial location of electrodes carried by the lead, such as lead 114 or any leads described herein.

Processing circuitry 210 then delivers the AEF therapy by delivering a first electric field from a first electrode combination (604) alternating with delivery of a second electric field from a second electrode combination different than the first electrode combination (606). For example, the first and electrode combinations may include electrodes from multiple cylindrical leads implanted in tissue adjacent to, and around, the resection cavity. In some examples, the first and second electrical fields may be phase shifted so as to not overlap. In other examples, the first and second electrical fields may be phase shifted so as to partially overlap in time. In some examples, the first and second electrical fields may be temporally interleaved to be fully non-overlapping or partially overlapping. Although the first and second electrical fields may be delivered with the same amplitude and frequency, the first and second electrical fields may be defined by different amplitudes and/or different frequencies (e.g., 150 kHz and 200 kHz). The first and second electrode combinations may use completely different electrodes or partially different electrodes, for example. The first and second electrode combinations may be selected to generate respective electrical fields that are orthogonal to each other or oblique, in some examples. Although all electrodes of the first and second electrode combinations may be implanted in some examples, one or more of the electrodes may be external electrodes in other examples. In general, processing circuitry 210 may determine the frequency of electrical field alternation based on the number of electrical combinations used to alternate the electrical fields for therapy. No interphase period may be required between the delivery of each electric field, although processing circuitry 210 may provide an interphase period in some examples.

Processing circuitry 210 then determines whether to terminate the AEF therapy (608). If processing circuitry 210 determines that AEF therapy is not to be terminated, processing circuitry 210 continues to deliver the first and second electric fields (604 and 606). If processing circuitry 210 determines that AEF therapy is to be terminated or otherwise paused, processing circuitry 210 stops delivering the AEF therapy to the patient (610).

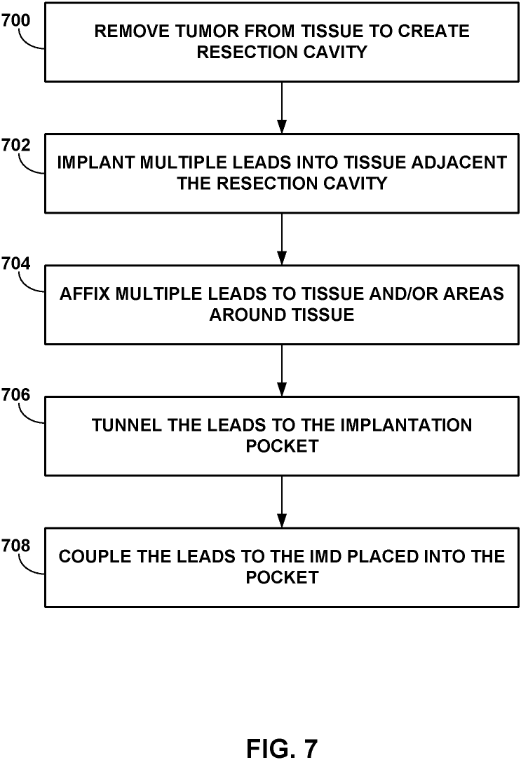

FIG. 7 is a flowchart illustrating an example technique for implanting multiple leads for delivering AEF therapy to a patient. As shown in the example of FIG. 7, a clinician may surgically remove a tumor, such as a glioblastoma, from tissue to create a resection cavity (700). The tissue may be a portion of the brain that may be superficial and near the skull or more deep from the skull. The resection cavity is generally a void of tissue, but may collapse over time or fill with fluid, such as cerebral spinal fluid (CSF). In any event, some tumor cells may be remaining in the tumor bed, or tissue adjacent the resection cavity, that remains after the tumor is removed. Therefore, the patient may benefit from subsequent AEF therapy, for example, that can reduce the likelihood that any remaining tumor cells will proliferate and create another tumor and/or metastasize over time.

During the same surgical procedure, the clinician may then implant multiple implantable medical leads into the tissue adjacent to the resection cavity (702). Since a portion of the skull is already to removed to extract the tumor, the clinician already has access to the remaining tissue in the example of a tumor being removed from the brain. The multiple leads may be inserted at spaced apart locations around the periphery of the resection cavity in order to enable the AEF therapy to reach any tumor cells that remain in the tissue surrounding the resection cavity. In some examples, the clinician may use one or more implantation plates or other devices described herein to facilitate lead insertion and or improve lead retention over time. In some examples, the clinician may first insert a bent stylet or pre-curved introducer that can be used to place a normally straight lead into a curved configuration to target tissue, such as around the curved form of a resection cavity.

Once inserted to the target tissue, the clinician can affix the multiple leads to tissue and/or area around the tissue to which the leads are implanted (704). In some examples, the clinician may use a single securing method for each lead or more than one securing method in other examples. For example, a clinician may utilize a primary and secondary fixation mechanism for each lead. A primary fixation method may include adhering a portion of each lead to a respective portion of the tissue into which the lead has been inserted. For example, applying an adhesive between the lead and a surface of the brain may be a primary fixation method. A secondary fixation method may include clamping or otherwise securing the lead to the skull using a fixation structure. These secondary fixation structures may include dog bones, straps, screws, friction fit devices, stretchable members, etc. The secondary fixation may enable strain relief of extra length of lead (e.g., curved portions of the lead or one or more strain relief loops) between the primary fixation and the secondary fixation to reduce the risk of lead stretch or tissue pulling with lead movement. In some examples, secondary fixation may be accomplished with bone cement, glue, or other adhesive.

Once the leads are affixed to tissue, the clinician can tunnel the leads to the implantation pocket where the IMD will be disposed (706). The implantation pocket may be a void created remote from the target tissue for therapy, such as a subcutaneous or submuscular location near the clavicle in the example of the brain being the target tissue. The clinician then can electrically and mechanically couple the leads directly to, or via one or more lead extensions, the IMD that will be placed in the implantation pocket (708). The clinician can then program and then start delivery of AEF therapy or other electric field therapy.

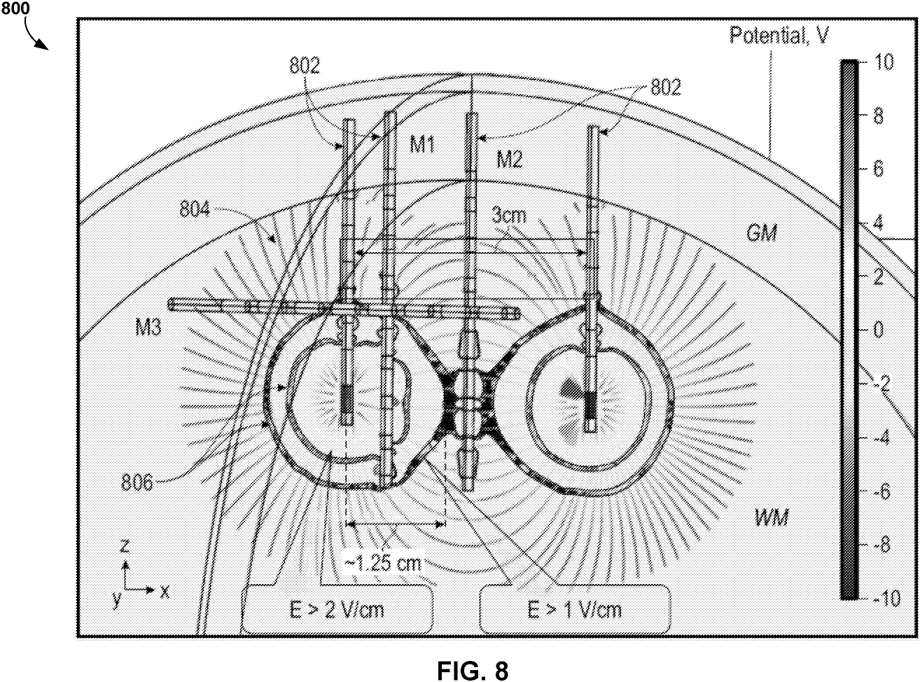

FIG. 8 is a conceptual diagram of example electrical fields generated between implanted leads. As shown in the example of FIG. 8, simulation 800 illustrates how electrical fields 804 can be generated in tissue from all or subsets of leads 802 implanted within the patient. Boundaries 806 indicate respective electric field densities at those locations at respective distances from the respective lead. For example, the inner boundary of boundaries 806 indicates that energy is greater than 2 V/cm within that boundary, and the outer boundary of boundaries 806 indicates that the energy is still greater than 1 V/cm. In some examples, leads separated by 3 cm may still generate a field strength greater than 1 V/cm at distances within 1.25 cm from each lead. Different electrode sizes and tissue conductivity may change these field strengths or electric field gradients for other subjects.

Using such a simulation, system 100 can suggest lead spacing or other implantation details for each lead of leads 802 and/or other parameter values such as frequencies or amplitudes of the electric fields in order to affect the desired tissues. In some examples, simulation 800 may account for the resection cavity and how the electric field will or will not propagate through CSF fluid or other material within the resection cavity. In some examples system 100 may consider that fluid shunting may increase power demands or determine any other factors as part of the simulation for therapy planning.

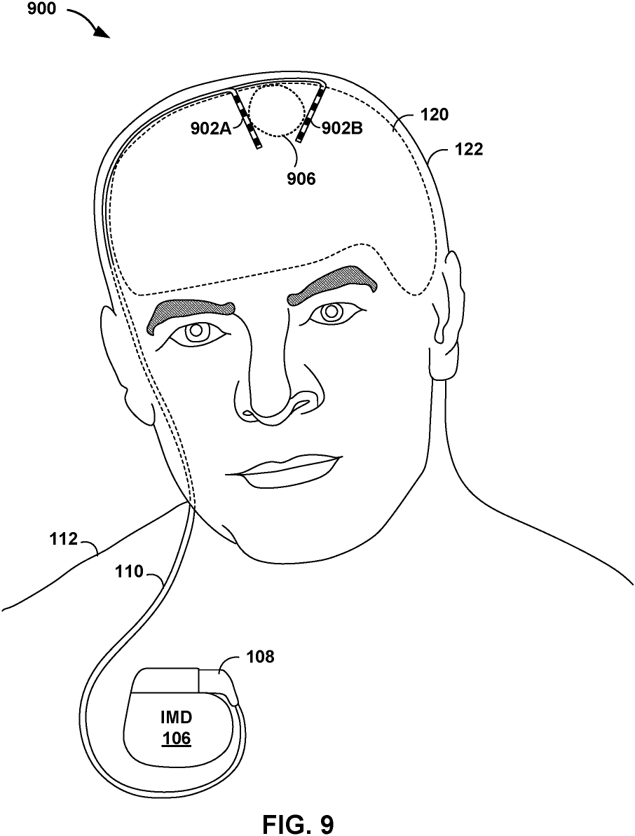

FIG. 9 is a conceptual diagram illustrating an example multiple pronged lead configured to deliver alternating electric field (AEF) therapy. As shown in the example of FIG. 9, system 900 includes IMD 106 couped to leads 902A and 902B (collectively “leads 902”) via lead extension 110. System 900 may be an example of system 100 described herein, and leads 902 may be examples of one of leads 114 described herein. Leads 902 are cylindrical leads with four ring electrodes each, but other types of leads may be used in other examples. Also, two leads 902 are shown, but three or more leads may be implanted in brain 120 in other examples. For example, IMD 106 may be coupled to four leads, six leads, or any other number of leads spaced out in tissue adjacent resection cavity 906 and/or at least partially within resection cavity 906.

Leads 902 are implanted in a slanted configuration such leads 902 are not parallel to each other. Generally, leads 902 are implanted within brain 120 and adjacent resection cavity 906 such that all electrodes of leads 902 are disposed within portions of brain 120. This configuration may be selected in order to prevent current interaction with CSF that may disrupt the electric field propagation through the remaining tissue around resection cavity 906. However, in some examples, one or more electrodes of leads 902A and/or 902B may be placed within resection cavity 906 in order to achieve a desired electric field. In some examples, one electrode in contact with CSF may cause electrical current to spread to create tissue area adjacent resection cavity 906. In any case, the electrodes carried by leads 902 may be configured to produce an electric field in response to IMD 106 driving electrical current through the respective electrodes. In some examples, the electrodes of leads 902 may be used to deliver AEF therapy or some other electrical field modulation therapy. The electric fields from each electrode may be alternating in time in order to produce an effect similar to AEF therapy described herein in order to provide various treatments, such as reducing or preventing cell division for treating glioblastoma.

In the example of FIG. 7, leads 902 are shown as substantially linear, or straight for the portions carrying the electrodes. However, in other examples, leads 902 may have a curved distal end or be placed within a curved sheath or other structure that can maintain the electrode carrying distal portion of one or more leads 902 in a predetermined or clinician defined curvature. For example, the distal ends of leads 902 may be curved to follow the curvature of tissue adjacent resection cavity 906.

Processing circuitry of IMD 106 may control IMD 106 to deliver electric field therapy via implantable leads 902 disposed adjacent to resection cavity 906 of patient 112. In some examples, IMD 106 delivers the electric field therapy by delivering alternating electric fields between electrodes of each lead 902A and 902B. In other words, the electric fields will be generated between the electrodes of the same lead. In another example, IMD 106 delivers the electric field therapy by delivering alternating electric fields between electrodes of adjacent lead pairs within the plurality of implantable leads. In other words, the electric fields will be generated between electrodes of different leads. In another example, IMD 106 delivers the electric field therapy by delivering alternating electric fields between electrodes of lead pairs across the resection cavity. In this example, the electric fields may be generated between leads on opposing sides of the resection cavity. For multiple pairs of leads where each pair is across from each other over the resection cavity, IMD 106 may deliver the electric field therapy by interleaving first alternating fields between a first lead pair across the resection cavity from each other with second alternating fields between a second lead pair across the resection cavity from each other.

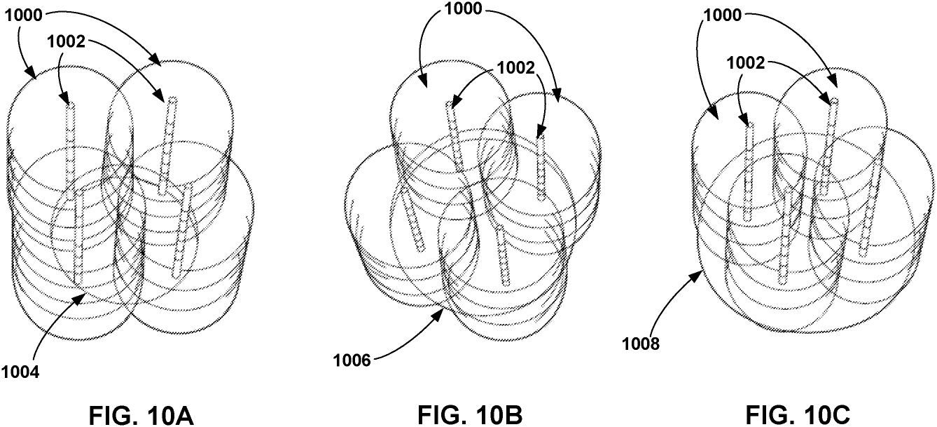

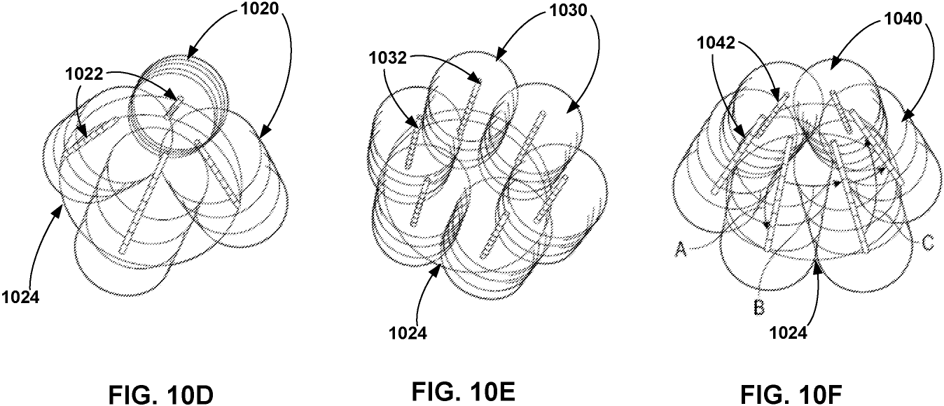

FIGS. 10A-10F are conceptual diagrams illustrating example lead configurations and corresponding electric fields between the leads. As shown in the example of FIG. 10A, four leads 1002 may be configured to generate electric fields 1000 that establish coverage of at least 1.0 V/cm that may fully cover a 3 cm diameter resection cavity 1004 or tumor bed. However, larger resection cavities, or tumor beds, may not be fully covered by four leads in this parallel column configuration. As shown in the example of FIG. 10B, four leads 1002 may be configured to generate electric fields 1000 that establish coverage of at least 1.0 V/cm for all or mostly all of the volume for a 4 cm diameter resection cavity 1006 or tumor bed. This may still be effective therapy. As shown in the example of FIG. 10C, four leads 1002 may be configured to generate electric fields 1000 that establish coverage of at least 1.0 V/cm for some of the volume of a 5 cm diameter resection cavity or tumor bed, but a substantial portion of the resection cavity or tumor bed may not receive effective therapy by leads 1002. In these examples, all of the leads may be implanted parallel to each other and provide electric field coverage over the target tissue (e.g., the tumor bed) surrounding the resection cavity. However, these examples show that this parallel lead configuration using four leads 1002 may not sufficiently treat all of the resection cavity or tumor bed such as in the example of resection cavity 1008 of FIG. 10C. Increasing the number of leads, changing the lead spatial configuration, or other approaches may increase the reach of electric fields to the target tissue volume.

As shown in the example of FIG. 10D, four leads 1022 may be configured to generate electric fields 1020 that can establish coverage of at least 1.0 V/cm for most of a 5 cm diameter resection cavity, and leads 1022 are implanted at an angle to each other in a pyramidal form. However, as shown, electric fields 1020 do not quite cover all of resection cavity 1024. For some larger recession cavities, more leads may be needed to provide effective electric field coverage. As shown in the example of FIG. 10E, six leads 1032 may be configured in a generally parallel and circle manner to generate electric fields 1030 that can establish coverage of at least 1.0 V/cm for a 5 cm diameter resection cavity 1024. As shown in the example of FIG. 10F, six leads 1042 may be configured to generate electric fields 1040 that can establish coverage of at least 1.0 V/cm for a 5 cm diameter resection cavity 1024, with the leads are implanted at angles to each other in a pyramidal form. In some examples, the strength of electric field diminishes over distance, so larger volume resection cavities may require a greater number of leads to provide effective electric field strength at all of the target tissue adjacent the resection cavity. For example, tumors or resection cavities less than 5 cm in diameter may be treated using 4 or fewer leads. Tumors or resection cavities of 5 cm or greater in diameter may benefit from 6, 7, or even more leads to treat all tissue with the electric fields. Tilting the leads with respect to each other may enable improved coverage for some tissue areas. For larger numbers of leads, the AEF therapy may be configured to switch between different possible electric field vectors.

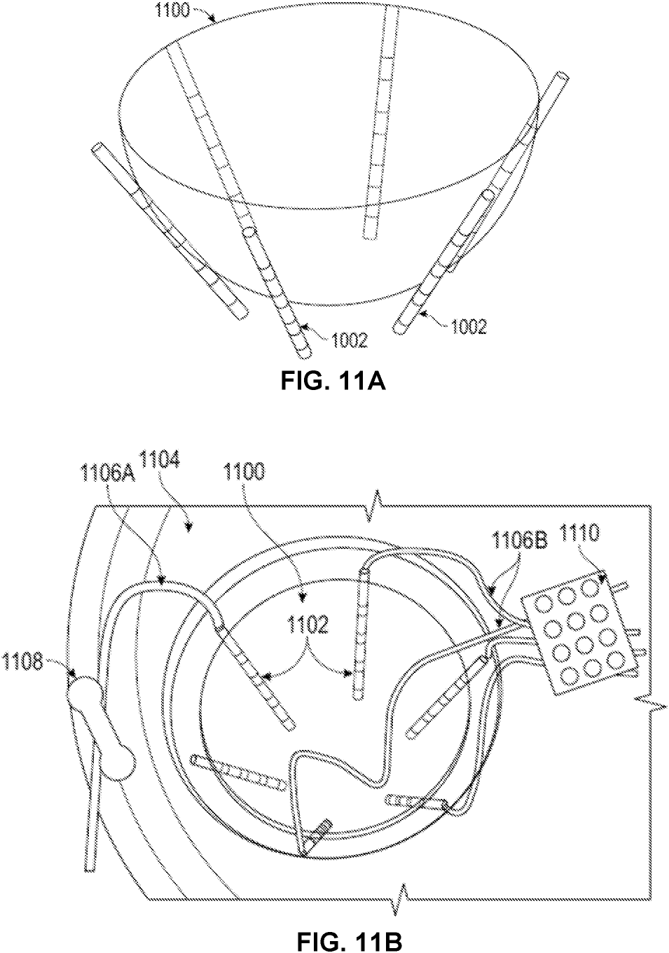

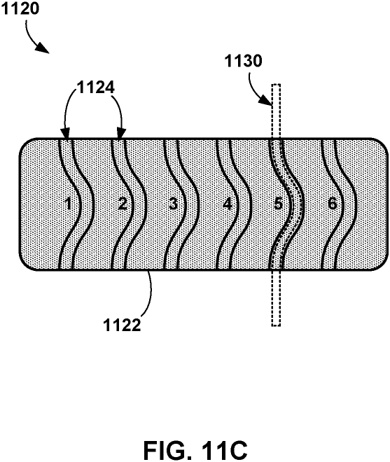

FIGS. 11A and 11B are illustrations of an example set of implantable leads implanted with respect to a resection cavity. As shown in the example of FIG. 11A, six leads 1002 are implanted in an angled and spaced pattern around the outside of resection cavity 1100. FIG. 11B illustrates a top view of the lead configuration of FIG. 11A. Resection cavity 1100 is within skull 1004. Leads 1102 are implanted in the tissue adjacent to resection cavity 1100. When implanting leads 1102 after removing the tumor, a clinician may secure each lead of the plurality of implantable leads 1102 to the patient via primary fixation to brain tissue and secondary fixation to a skull of the patient. In other words, implantable leads 1102 may be secured to tissue using multiple types of fixation to respective different tissue types.

An example of primary fixation includes an adhesive that is applied to a portion of each of leads 1102 and respective portions of the brain within skull 1104. Secondary fixation may include coupling each lead 1102 to skull 1104 via a fixation structure, such as dogbone 1108 or attachment mesh 1110. For example, lead 1106A may be captured against skull 1104 beneath dogbone 1108 that is attached to skull 1104 via screws, stables, adhesive, or any other fixation element. Similarly, leads 1106B may be captured against skull 1104 beneath mesh 1110 that is attached to skull 1104 via screws, stables, adhesive, or any other fixation element. Dogbone 1108 or attachment mesh 1110 may be ridged or flexible based on the fixation needs for the respective leads. Is some examples, slack may be maintained between the primary and secondary fixation locations to enable the brain to move without stressing either of the primary or secondary fixations.

FIG. 11C is a conceptual diagram of an example fixation structure 1120 for securing leads within tissue. As shown in the example of FIG. 11C, fixation structure 1120 may be attachable to the skull or other tissue location and configured to secure some or all of the leads implanted within the patient. Fixation structure 1120 may define a plurality of curved channels 1124 configured to create a friction fit between a respective lead, such as lead 1130, of the plurality of implantable leads and a channel surface of a respective channel 1124 of the plurality of curved channels. Baseplate 1122 may define all of channels 1124. The curvature of channels 1124 may be selected to have a small enough radius to create a friction fit between the lead and surface of the respective channel 1124 and large enough to prevent damage of the secured lead. Fixation structure 1120 may be glued, screwed, or otherwise attached to the skull or other desired securing location.

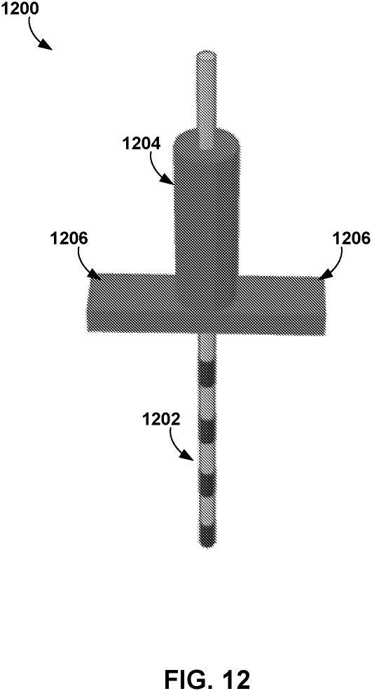

FIG. 12 is an example system that includes a lead 1202 (e.g., lead 114 or 1102) and spacer 1200 that facilitates implantation of lead 1202. Spacer 1200 includes a handle 1204 coupled to flanges 1206 that extend radially outward from the channel in spacer defined by handle 1204. Handle 1204 may be configured to be manipulated by a hand of the clinician. The radial distance of flanges 1206 may define the spatial distance, or target distance, between adjacent leads. Therefore, when lead 1202 is disposed within spacer 1200, the clinician can implant lead 1202 or other leads (such as leads 114 or 1102) by positioning lead 1202 at the target distance with respect to a second lead. In addition, the distal surface of flanges 1206 may be configured to contact the skull or brain and act as a depth stop for the insertion of lead 1202. Lead 1202 may be captured within the channel of spacer 1200 using friction fit, a removable clamp, or any other type of passive or active mechanism.

In some examples, flanges 1206 may have a length that is adjustable by the user. For example, flanges 1206 may be telescoping and set with a set screw. In other examples, flanges may have perforations at different distances that promote breaking of the end of the flange at the perforations when force is applied by fingers of the user. In some examples, the different distances of the perforations or telescoping options may be labeled, such as in 1 mm or 2 mm increments, to assist the user in achieving the desired length of each of flanges 1206. In some examples, once lead 1202 is implanted, the user may remove spacer 1200 from lead 1202. In other examples, handle 1204 may be detachable and removable from flanges 1206 and lead 1202 in order for flanges 1206 to remain with lead 1202 and maintain spacing to adjacent leads. In this example, the surgeon may apply a glue or other adhesive (e.g., surgicell that builds a scaffold) to the distal surface of flanges 1206 to adhere flanges 1206 to the surface of the tissue, such as the brain or skull.

In some examples, spacer 1200 may have a predetermined non-orthogonal angle of the channel for lead 1202 relative to the distal surface of flanges 1206 that can facilitate angled insertion of lead 1202. In other examples, the angle of the channel may be adjustable by a pivot or rotatable portion of handle 1204 with respect to flanges 1206. In other examples, the channel through handle 1204 may be larger than the diameter of lead 1202 or conical or double conical in shape to enable the clinician to insert lead 1202 at different angles with respect to the surface of flanges 1206.

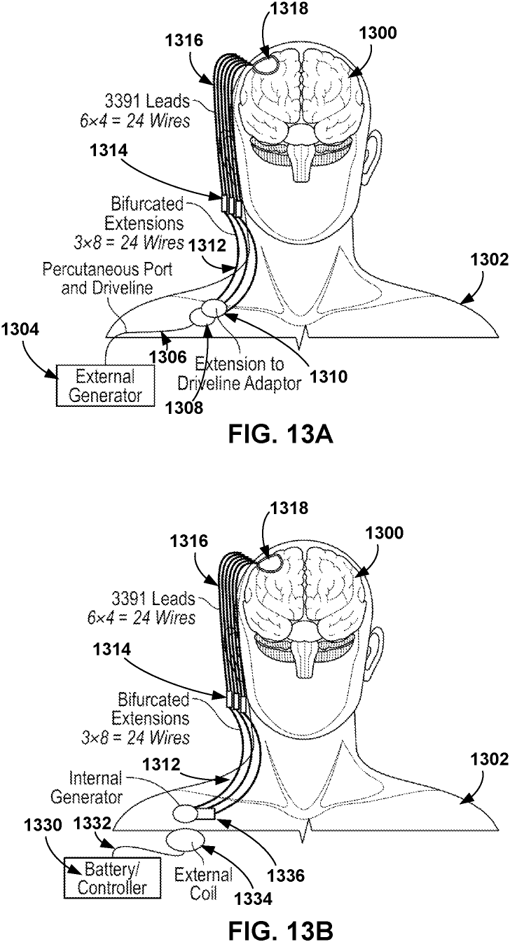

FIGS. 13A and 13B are conceptual diagrams illustrating example systems for powering the delivery of AEF therapy. In the example of FIG. 13A, leads 1316 are coupled to bifurcated extensions 1312 via respective connectors 1314. Bifurcated extensions 1312 are couped to an extension 1310 that is implanted within patient 1302. Extension 1310 is coupled to adapter 1308, and adapter 1308 is connected to external generator 1304 via a percutaneous driveline configured to be placed through a percutaneous port and directly connected to external generator 1304. In this configuration, external generator 1304 provides power via wire to the implanted leads (e.g., leads 1102) so that electrodes carried by respective leads 1316 can generate electric fields to target tissue 1318 (e.g., tumor or resection cavity).

In the example of FIG. 13B, which is similar to FIG. 13A, leads 1316 are coupled to bifurcated extensions 1312 via respective connectors 1314. Bifurcated extensions 1312 are couped to an internal generator 1336 that is implanted within patient 1302, such as IMD 106. The battery of internal generator 1336 may be charged internal generator 1336 may be directly powered from energy delivered by an external coil 1334 powered by external power device 1330 via cable 1332. This energy transfer may be effected using inductive energy transfer (e.g., magnetic field produced by external coil 1334 which induces a current into an internal coil within or directly connected to internal generator 1336.

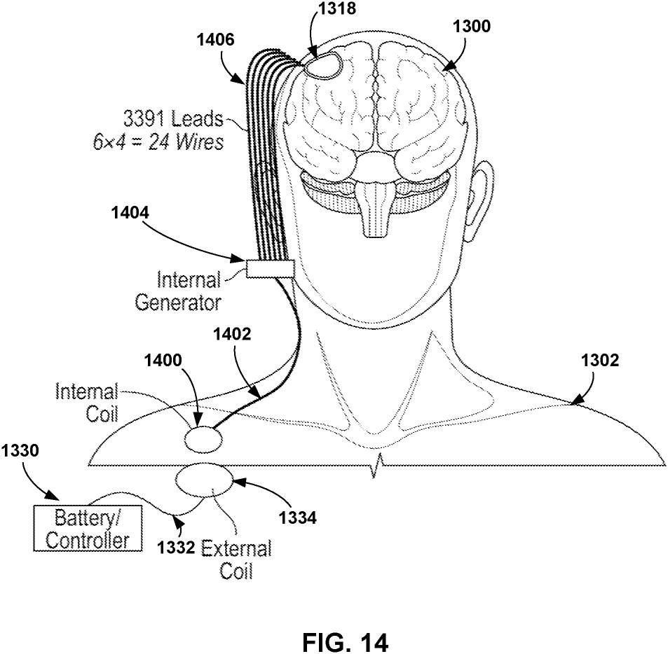

FIG. 14 is a conceptual diagram illustrating an example system powering the delivery of AEF therapy. The example of FIG. 14 may be similar to FIGS. 13A and 13B. However, in the example of FIG. 14, leads 1406 are coupled directly to internal generator 1404 (e.g., IMD 106) which generates the signals for electric fields produced by electrodes carried by respective leads 1406 to resection cavity 1318 (or tumor bed) in brain 1300. Internal generator 1404 may be powered directly from external power wirelessly, so an internal coil 1400 extends from internal generator 1404 via cable 1402. The internal coil 1400 can receive wireless energy (e.g., via inductive coupling) from an external coil 1334 powered, via cable 1332, by an external power device 1330, such as a battery charger or controller. In this manner, internal generator 1404 may be configured to receive operational power wirelessly from external power device 1330. In some examples, electrical fields may be generated using multiple types of power, such as a rechargeable battery within internal generator 1404 and direct power from external sources. In this manner, internal generator 1404 may be coupled to two different coils, one configured to receive power to recharge a rechargeable battery and a second coil configured to receive operational power that can directly generate the electrical fields via the leads.

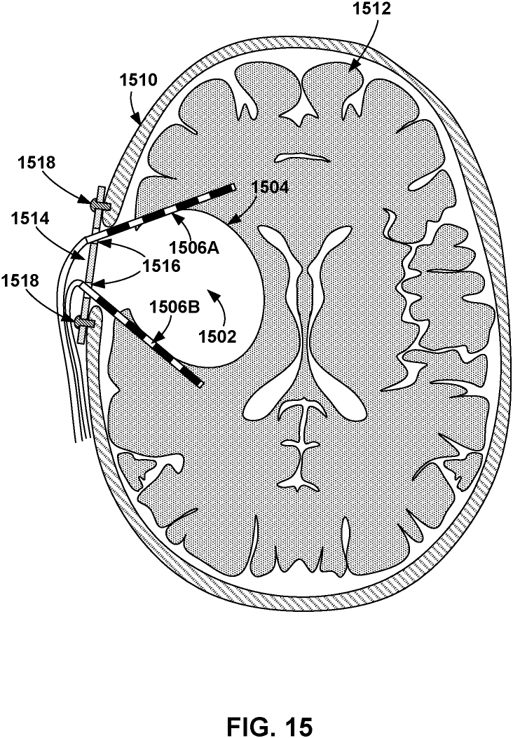

FIG. 15 is a cross-sectional view of an implantation plate 1514 configured for implanting leads 1506A and 1506B (collectively “leads 1506”) around resection cavity 1502. As shown in FIG. 15, brain 1512 is located within cranium 1510. Resection cavity 1502 may be formed by removing a tumor, for example. Resection cavity 1502 may also be described as or part of a tissue resection region at which electrodes can be implanted. Although only two leads 1506 are shown, three, four, five, six, or more leads may be implanted around resection cavity 1502. In some examples, all of leads 1506 may be implanted through at least a portion of tissue 1504 defining resection cavity 1502. In some examples, each electrode of each implantable lead 1506 is surrounded by tissue. In other examples, at least one electrode carried by the plurality of implantable leads 1506 is positioned within resection cavity 1502. This situation may occur when a lead passes through a superficial portion of tissue, then through an edge of resection cavity 1502, and then again may be inserted through a deep portion of tissue.

Implantation plate 1514 may assist in the placement of leads 1506. Implantation plate 1514 defines guide channels 1516 through which respective leads 1506 can be inserted into brain 1512. Screws 1518 can fix implantation plate 1514 to the exterior of skull 1510. In this manner, implanting implantable leads 1506 includes inserting each implantable lead 1506 through respective guide channels 1516 of implantation plate 1514 configured to contact skull 1510 and remain external of resection cavity 1502. In some examples, implantation plate 1514 defines at least two guide channels 1516. In other examples, implantation plate 1514 defines at least four guide channels 1516 for respective leads. In some examples, implantation plate 1514 is configured such that a user can remove implantation plate 1514 after securing implantable leads 1506 to tissue, such as brain 1512 and/or skull 1510. As shown in the example of FIG. 15, the clinician may attach implantation plate 1514 to skull 1510 of the patient using a fixation mechanism, such as screws 1518, adhesive, etc.

Implantation plate 1514 may be ridge, semi-rigid, or flexible in one or more dimensions. For example, implantation plate 1514 may be constructed of a metal such as stainless steel or titanium, a polymer, a composite, or any other biocompatible material. In some examples, implantation plate 1514 may be constructed of a bioresorbable material that degrades over time when leads 1506 are encapsulated by tissue.

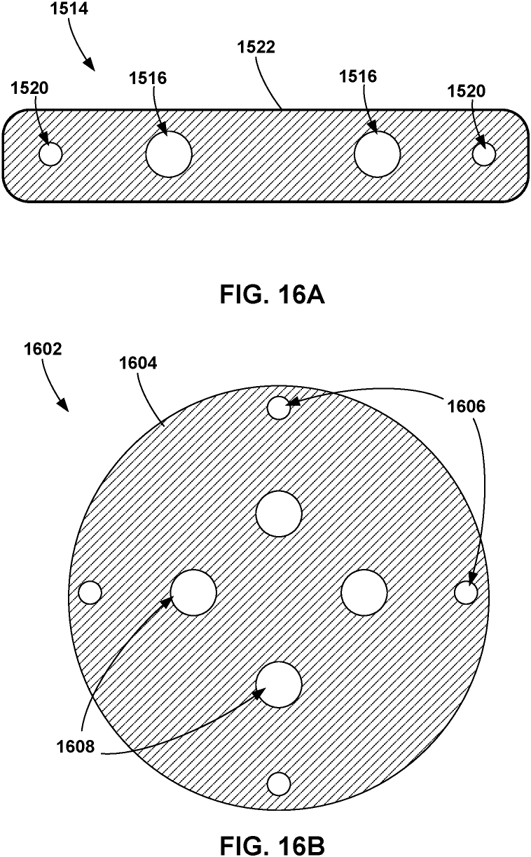

FIGS. 16A and 16B are top views of example implantation plates 1514 and 1602 for implanting leads around a resection cavity. The cross-sectional view of implantation plate 1514 is shown in FIG. 15. As shown in FIG. 16A, implantation plate 1514 includes structure 1522 that defines two guide channels 1516 configured to pass respective implantable leads and fixation channels 1520 configured to accept fastening devices such as bone screws. Implantation plate 1514 may have a length configured to span an opening in a skull such that fixation channels 1520 remain over the skull and guide channels 1516 remain over the opening in the skull. In some examples, the walls that define guide channels 1516 may be orthogonal to the top surface of structure 1522. In other examples, at least some portions of the walls that define guide channels 1516 may be oblique to the top surface of structure 1522 to define a center axis that is also oblique to the top surface such that the lead is guided through structure 1522 at a non-orthogonal angle.

Implantation plate 1602 shown in FIG. 16B may be similar to implantation plate 1514. However, implantation plate 1602 may be circular or otherwise configured to cover the entire opening in the skull through which leads will be implanted. Implantation plate 1602 includes structure 1604 that defines fixation channels 1606 configured to accept respective fastening devices such as bone screws. Four fixation channels 1606 are shown, but fewer or greater fixation channels 1606 may be used in other examples. Structure 1604 also defines fixation channels 1608 configured to accept respective implantable leads and guide them into respective positions around a resection cavity. Similar to guide channels 1516, guide channels 1608 may have walls orthogonal to the top surface of structure 1604 or oblique to promote orthogonal or non-orthogonal placement of the leads. Although four guide channels 1608 are shown in the example of FIG. 16B, fewer or greater numbers of guide channels 1608 may be used in other examples. In some examples, structure 1604 may define a grid or pattern of a plurality of guide channels of a greater number than leads intended to be implanted. The grid of guide channels 1608 may be spaced in a circular pattern, multiple nested circle patterns, or random locations such that guide channels 1608 can be at different circumferential and different radial positions. In this manner, the clinician may select which guide channels to use for implantation while at least some of the guide channels remain unused. Although implantation plate 1602 is circular in shape, implantation plate 1602 may be rectangular, square, triangular, or any other shape. In some examples, the different guide channels 1608 may be configured in pairs such that pairs of leads can be assigned to respective guide channels to insert the leads at appropriate distances to create desired electric fields for AEF or other therapies. In some examples, a curved or straight polymer sleeve (e.g., silicone, etc.) may be used for guiding and/or maintaining the leads into the appropriate locations.

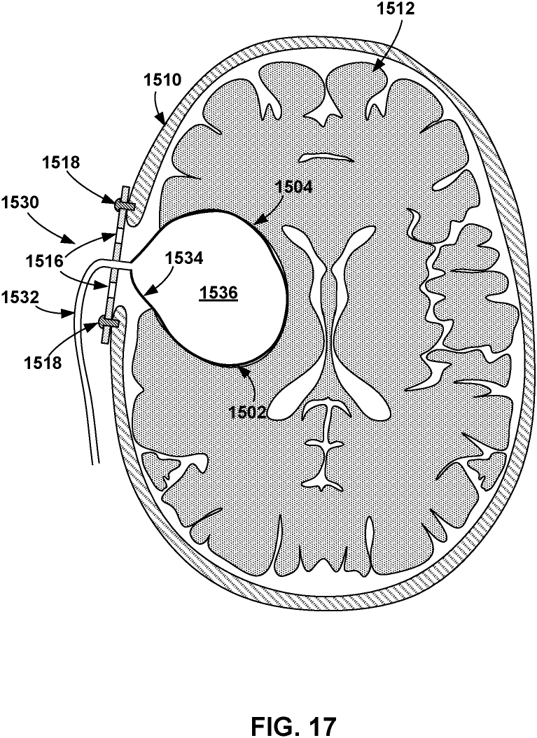

FIG. 17 is a cross-sectional view of an implantation plate 1530 including expanding device 1534 configured to fill resection cavity 1502 during lead implantation. Implantation plate 1530 may be similar to implantation plate 1514 of FIG. 15. However, expandable member 1534 may be attached to implantation plate 1530 through an opening in implantation plate 1530. As shown in the example of FIG. 17, expanding device 1534 is a stretchable material similar to a balloon. Expanding device 1534 may be coupled to tubing 1532 that is in fluid communication with expanding device 1534 and a fluid source configured to deliver fluid or gas to expanding device 1534.

Expanding device 1534 may be configured to maintain the shape and/or size of resection cavity 1502 to facilitate insertion of leads into the tissue around resection cavity 1502 while reducing the risk that the tissue 1504 surrounding resection cavity 1502 would collapse during lead insertion. Fluid 1536 used to pressurize and inflate expanding device 1534 may include saline, water, or even a fluid comprising radiopaque or otherwise visible elements for imaging. In other examples, fluid 1536 may be a gas such as air or nitrogen. The walls of expanding device 1534 may be flexible and configured to conform to tissue 1504 surrounding resection cavity 1534. Therefore, a clinician may expand expanding device 1534 within resection cavity 1502 and then insert each implantable lead into tissue while expanded device 1534 is expanded within resection cavity 1502. In other examples, expanding device 1534 may not use internal pressure to expand. Instead, expanding device 1534 may include an expandable lattice of struts, memory material (e.g., polymer or nitinol) that creates a volumetric structure within resection cavity 1502, or any other structure that can fill portions of resection cavity 1502 and reduce the movement of tissue 1504 into resection cavity 1502. In this manner, it may be easier for the clinician to insert leads into tissue adjacent resection cavity 1502. After inserting the leads (e.g., as shown in FIG. 15), the clinician may collapse expanding device 1534 and remove from the patient. In some examples, expanding device 1534 may be left in place with the leads for therapy. Balloon may provide insulation that may facilitate electric fields affecting surrounding tissue. In other examples, an insulation material, such as cellulose, adhesive, gammalite, or other non-electrically conductive material may be placed within the resection cavity to reduce current leakage across CSF in the resection cavity.

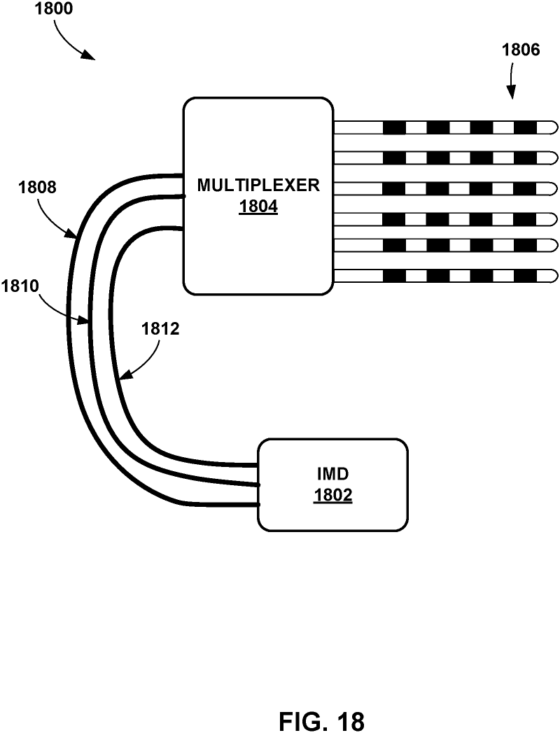

FIG. 18 is a conceptual diagram of an example system 1800 that includes an implantable medical device 1802 and multiplexer 1804 to control AEF therapy. IMD 1802 may be similar to IMD 106, but IMD 1802 may be configured to couple to fewer conductors than the number of leads 1806. Six leads 1806 are shown in the example of FIG. 18, but more or fewer leads may be used in other examples. Each of leads 1806 may be coupled to multiplexer 1804. Multiplexer 1804 may be configured to include separate contacts for each contact associated with each electrode on each of leads 1806. In this manner, multiplexer 1804 may support independent control of each electrode. In other examples, multiplexer 1804 may include contacts that combine or gang together two or more electrodes of a single lead and/or combine or gang together electrodes of multiple leads. For example, a contact within multiplexer 1804 may be configured to create a single electrical connection to multiple contacts of leads 1806 such that the electrodes associated with the multiple contacts function as a single electrode. Ganging together electrodes in this manner may reduce the number of controllable electrode combinations, but may reduce the size of multiplexer 1804. Since the desired electric fields may not require full independent control of all electrodes, combining electrodes may maintain the desired electric fields. Although each of leads 1806 are shown to include four electrodes each, each lead may include fewer or greater number of electrodes in other examples. In some examples, multiplexer 1804 may have more connection ports for leads than leads that are used. In this manner, the clinician can add additional leads to get to seven, eight, or more leads during the procedure or at a later time.

Conductors 1808, 1810, and 1812 may couple IMD 1802 to multiplexer 1804. Conductors 1808, 1810, and 1812 may be contained within separate leads or combined into a single lead or cable. Conductors 1808 and 1810 may carry current to and from leads 1806 to provide the electric fields. Additional conductors may be provided to support simultaneous different electric fields, or different electrical stimulation therapies (e.g., AEF therapy and electroporation therapy) in other examples. Conductor 1812 may be configured to carry control signals to multiplexer 1804 so that IMD 1802 can control multiplexer 1804 to deliver the desired electrical fields between the desired sets of electrodes carried on leads 1806. In some examples, operational power for multiplexer 1804 may be obtained via any of conductors 1808, 1810, or 1812, or another conductor may be included to carry power for operating the switches within multiplexer 1804. In some examples, five conductors may be used in total, such as 2 control conductors, a ground conductor, and two stimulation lines that provide the electrical signals that produce the electric fields. System 1800 may enable IMD 1802 to control multiplexer 1804 that is separate from IMD 1802 to select subsets of leads 1806 for respective electric fields of the electric field therapy.

System 1800 enables IMD 1802 to be implanted remote from leads 1806 without connecting each lead directly to a header of IMD 1802. In this manner, multiplexer 1804 can be provided to connect to any number of leads 1806 and gang together, if desired, any combination of electrodes from leads 1806, without physical modification of multiplexer 1804. In addition, the fewer conductors that connect IMD 1802 to multiplexer 1804 reduces the size of the lead or cable that needs to be tunneled through tissue from IMD 1802 to multiplexer 1804.

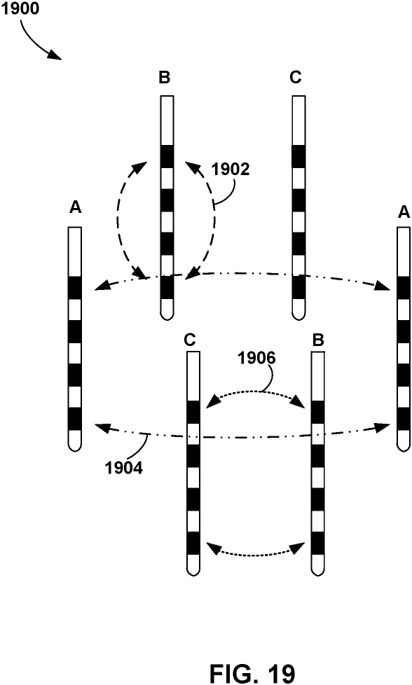

FIG. 19 is a conceptual diagram of example alternating electric fields between an example array of leads 1900. Leads 1900 may be similar to leads 1102 or leads 1806. Leads 1900 may be arranged in a volumetric or three-dimensional array such as around a resection cavity. In this spatial arrangement, different types and locations of alternating electric fields may be delivered as desired. For example, the system may deliver electric fields 1902 that alternates between proximal and distal electrodes of the same lead, such as lead B or any or all of leads 1900. In another example, the system may deliver electric fields 1904 that alternate between one or more electrodes of leads A and A that are arranged across the resection cavity from each other. In other examples, the system may deliver electric fields 1906 that alternate between electrodes of adjacent leads, such as leads B and C of the arrangement of leads 1900. Some leads may be ganged together, or certain electrodes from different leads, such that the operate as a single electrode. For example, leads A may be ganged together, leads B may be ganged together, and leads C may be ganged together. Therefore, when used to deliver an electric field, electrodes of leads A may be cathodes and electrodes of leads C may be anodes, for example, to create electric fields that can alternate between these sets of leads.

Any of the electric fields in FIG. 19 may be delivered between different pairs of leads throughout the arrangement of leads 1900 in a simultaneous and/or interleaving manner. In some examples, the system may cycle through different types of electric fields 1902, 1904, and/or 1906. These example electric fields are provided for illustration only, and additional electric fields may be provided between any combination of electrodes as supported by an IMD that is coupled to leads 1900. In some examples, leads 1900 may be configured such that adjacent neighboring leads are unique, which is shown in the A-B-C configuration of leads. Any non-repeating tiling patterns of leads and/or electrodes may also be used in some examples.

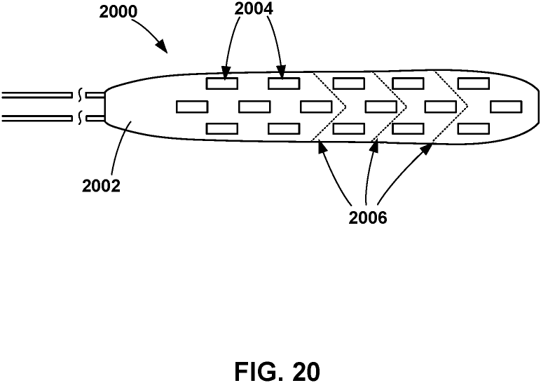

FIG. 20 is a conceptual diagram of an example paddle lead 2000 configured to be cut to a desired size for a patient. As shown in the example of FIG. 20, paddle lead 2000 may include housing 2002 and an array of electrodes 2004. Conductors within housing 2002 may be coupled to respective electrodes 2004. Housing 2002 may be constructed of a flexible material, such as a polymer, to enable paddle lead 2000 to conform to a surface of a resection cavity or otherwise be placed within or adjacent a resection cavity.

Housing 2002 may also include perforations 2006. Instead of perforations 2006, lines or other markings may be provided in other examples. Perforations 2006 may be provided to guide a user to cutting off a portion of housing 2002, and electrodes 2004, that are not needed for implantation. For example, if paddle lead 2000 is too long, a user may use scissors or other cutting device to cut along the distal perforations 2006 to cut off the distal end of housing 2002 and remove the distal three electrodes of electrodes 2004. Instead of perforations 2006, housing 2002 may not include any markings, and the clinician may simply remove any electrodes or portions of housing 2002 desired. The clinician may then implant paddle lead 2000 that has had the portion removed. Although removal of this portion of housing 2003 may also sever the conductors that were connected to the distal three electrodes, these exposed conductors may not reduce the effectiveness of paddle lead 2000. In some examples, the cut conductors may slightly pull back within housing 2002 to prevent them from being exposed to tissue. In other examples, the cut conductors may be exposed, but the surface area of the conductors exposed to tissue may be so small that any current may not substantially affect any desired electric field. In some examples, electric current associated with the cut conductors may improve the delivery of certain electric fields for therapy. In some examples, IMD 106 or other IMD coupled to paddle lead 2000 may identify any conductors that were cut or severed and prevent those conductors from being selected as part of electrodes for therapy. For example, a lead integrity test may be performed to identify impedances of electrodes, and cut conductors would appear has high impedances that may exceed a threshold. The system can the remove these high impedance electrodes/conductors from being selected as part of an electrode configuration for delivering electric fields.

Instead of a conformable paddle lead 2000, a gel film or other conformable structure with array of electrodes that can be placed on the surface of tissue defining the resection cavity. In some examples, additional depth leads may be inserted through holes in the gel film to provide electrodes into the tissue defining the resection cavity. The holes may provide paired holes for respective leads to create an alternating electric field between each lead.

A variety of different therapies are described herein that are related to each other, but some are slightly different. Generally, electric and magnetic stimulation therapy covers all of the therapies described herein. This includes, for example, direct current stimulation (DCS). Electric field therapy includes alternating current stimulation, which also includes alternating electric field (AEF) therapy, which includes tumor treating field (TTF) therapy (e.g., AEF therapy within a range of 100 kHz to 500 kHz). Electric field therapy also includes pulse electric fields, which includes nanosecond pulsed electric fields (or nanopulse stimulation), which includes both irreversible electroporation and reversible electroporation. Electric and magnetic stimulation therapy also includes magnetic field therapy, which includes examples such as alternating magnetic field (AMF) therapy, oscillating magnetic field (OMF) therapy, and extremely low frequency electromagnetic field (ELF-EMF) therapy. Other types of therapy may also be included within any of these example categories of therapies.

The following examples are described herein.

Example 1: A system includes memory configured to store parameters defining electric field therapy; and processing circuitry configured to control an implantable medical device to deliver electric field therapy according to the parameters by at least alternating electric fields between electrodes of adjacent lead pairs of a plurality of implantable leads disposed adjacent to a resection cavity of a patient.

Example 2: The system of example 1, wherein the processing circuitry is configured to control the implantable medical device to deliver the electric field therapy by at least controlling the implantable medical device to deliver alternating electric fields between electrodes of each lead of the plurality of implantable leads.

Example 3: The system of any of examples 1 or 2, wherein the processing circuitry is configured to control the implantable medical device to deliver the electric field therapy by at least controlling the implantable medical device to deliver alternating electric fields between electrodes of lead pairs across the resection cavity, the plurality of implantable leads comprising the lead pairs.

Example 4: The system of any of examples 1 through 3, wherein the processing circuitry is configured to control the implantable medical device to interleave first alternating fields between a first lead pair across the resection cavity from each other with second alternating fields between a second lead pair across the resection cavity from each other.

Example 5: The system of any of examples 1 through 4, wherein the plurality of implantable leads comprises at least six implantable leads, wherein each lead of the plurality of implantable leads comprises a plurality of electrodes.

Example 6: The system of any of examples 1 through 5, wherein the processing circuitry is configured to control the implantable medical device to deliver the electric field therapy by at least controlling a multiplexer separate from the medical device to select subsets of leads from the plurality of implantable leads for respective electric fields of the electric field therapy, wherein the plurality of implantable leads are coupled to the multiplexer, and wherein the multiplexer is coupled to the implantable medical device via few conductors than the plurality of implantable medical leads coupled to the multiplexer.

Example 7: The system of any of examples 1 through 6, further comprising the implantable medical device, and wherein the implantable medical device is configured to receive operational power wirelessly from an external power device.

Example 8: The system of any of examples 1 through 7, wherein the plurality of implantable leads are configured to be implanted through at least a portion of tissue defining the resection cavity.

Example 9: The system of example 8, wherein the plurality of implantable leads are configured to be implanted such that each electrode of each implantable lead of the plurality of implantable leads is surrounded by the tissue defining the resection cavity.

Example 10: The system of example 8, wherein the plurality of implantable leads are configured to be implanted such that at least one electrode carried by the plurality of implantable leads is positioned within the resection cavity.

Example 11: The system of any of examples 8 through 10, further comprising a spacer, wherein the plurality of implantable leads are configured to be positioned such that a first lead of the plurality of implantable leads is positioned with respect to a second lead of the plurality of implantable leads via a spacer through which the first lead is inserted, wherein the spacer comprises at least one flange extending radially outward from a channel of the spacer through which the first lead is disposed, the at least one flange extending radially outward a distance corresponding to a target distance between the first lead and the second lead.

Example 12: The system of any of examples 8 through 11, further comprising an implantation plate configured to contact the skull and remain external of the resection cavity, wherein each of the implantable leads are configured to be inserted through a guide channel of an implantation plate.

Example 13: The system of example 12, wherein the implantation plate comprises at least two guide channels.

Example 14: The system of example 12, wherein the implantation plate comprises at least four guide channels.

Example 15: The system of any of examples 12 through 14, wherein the implantation plate is configured to be removed after securing the plurality of implantable leads to the patient.

Example 16: The system of any of examples 12 through 15, wherein the implantation plate is configured to be attached to a skull of the patient.

Example 17: The system of any of examples 8 through 16, further comprising primary fixation configured to secure each lead of the plurality of implantable leads to brain tissue of the patient and secondary fixation configured to secure each lead of the plurality of implantable leads to a skull of the patient.

Example 18: The system of example 17, wherein the primary fixation comprises an adhesive.

Example 19: The system of any of examples 17 and 18, wherein the secondary fixation comprises coupling each lead to the skull via a fixation structure.

Example 20: The system of example 19, wherein the fixation structure comprises a dog bone structure.

Example 21: The system of example 19, wherein the fixation structure comprises an attachment mesh.

Example 22: The system of example 19, wherein the fixation structure defines a plurality of curved channels configured to create a friction fit between a respective lead of the plurality of implantable leads and a channel surface of a respective channel of the plurality of curved channels.

Example 23: The system of any of examples 8 through 22, further comprising an expanding device configured to expand within the resection cavity, and wherein the plurality of implantable leads are configured to be inserted into tissue while the expanded device is expanded within the resection cavity.

Example 24: The system of any of examples 8 through 23, wherein at least one lead of the plurality of implantable leads configured to have a portion including one or more electrodes cut off from the remaining portion of the at least one lead, and wherein the remaining portion of the lead is configured to be implanted without the removed portion.

Example 25: The system of any of examples 1 through 14, wherein one or more electrodes of the plurality of electrodes comprises an electrode structure configured to conform to a surface of the resection cavity.

Example 26: A method includes controlling, by processing circuitry, an implantable medical device to deliver electric field therapy according to the parameters defining the electric field therapy by at least alternating electric fields between electrodes of adjacent lead pairs of a plurality of implantable leads disposed adjacent to a resection cavity of a patient.

Example 27: The method of example 26, wherein controlling the implantable medical device to deliver the electric field therapy comprises controlling the implantable medical device to deliver alternating electric fields between electrodes of each lead of the plurality of implantable leads.

Example 28: The method of any of example 26 or 27, wherein controlling the implantable medical device to deliver the electric field therapy comprises controlling the implantable medical device to deliver alternating electric fields between electrodes of lead pairs across the resection cavity.

Example 29: The method of any of examples 26 through 28, further comprising interleaving first alternating fields between a first lead pair across the resection cavity from each other with second alternating fields between a second lead pair across the resection cavity from each other.

Example 30: The method of any of examples 26 through 29, wherein the plurality of implantable leads comprises at least six implantable leads, wherein each lead of the plurality of implantable leads comprises a plurality of electrodes.

Example 31: The method of any of examples 26 through 30, wherein controlling the implantable medical device to deliver the electric field therapy comprises controlling a multiplexer separate from the medical device to select subsets of leads from the plurality of implantable leads for respective electric fields of the electric field therapy, wherein the plurality of implantable leads are coupled to the multiplexer, and wherein the multiplexer is coupled to the implantable medical device via few conductors than the plurality of implantable medical leads coupled to the multiplexer.

Example 32: The method of any of examples 26 through 31, further comprising receiving, by the implantable medical device, operational power wirelessly from an external power device.

Example 33: The method of any of examples 26 through 32, further comprising implanting the plurality of implantable leads through at least a portion of tissue defining the resection cavity.

Example 34: The method of example 33, wherein implanting the plurality of implantable leads comprises implanting the plurality of implantable leads such that each electrode of each implantable lead of the plurality of implantable leads is surrounded by the tissue.

Example 35: The method of example 33, wherein implanting the plurality of implantable leads comprises implanting the plurality of implantable leads such that at least one electrode carried by the plurality of implantable leads is positioned within the resection cavity.

Example 36: The method of any of examples 33 through 35, wherein implanting the plurality of implantable leads comprises positioning a first lead of the plurality of implantable leads with respect to a second lead of the plurality of implantable leads via a spacer through which the first lead is inserted, wherein the spacer comprises at least one flange extending radially outward from a channel of the spacer through which the first lead is disposed, the at least one flange extending radially outward a distance corresponding to a target distance between the first lead and the second lead.

Example 37: The method of any of examples 33 through 36, wherein implanting the plurality of implantable leads comprises inserting each implantable lead of the plurality of implantable leads through a guide channel of an implantation plate configured to contact the skull and remain external of the resection cavity.

Example 38: The method of any of examples 33 through 37, wherein the implantation plate comprises at least two guide channels.

Example 39: The method of any of examples 33 through 38, wherein the implantation plate comprises at least four guide channels.

Example 40: The method of any of examples 33 through 39, further comprising removing the implantation plate after securing the plurality of implantable leads to the patient.

Example 41: The method of any of examples 33 through 40, further comprising attaching the implantation plate to a skull of the patient.

Example 42: The method of any of examples 33 through 41, further comprising securing each lead of the plurality of implantable leads to the patient via primary fixation to brain tissue and secondary fixation to a skull of the patient.

Example 43: The method of example 42, wherein the primary fixation comprises an adhesive.

Example 44: The method of any of examples 42 and 43, wherein the secondary fixation comprises coupling each lead to the skull via a fixation structure.

Example 45: The method of example 44, wherein the fixation structure comprises a dog bone.

Example 46: The method of example 44, wherein the fixation structure comprises an attachment mesh.

Example 47: The method of example 44, wherein the fixation structure defines a plurality of curved channels configured to create a friction fit between a respective lead of the plurality of implantable leads and a channel surface of a respective channel of the plurality of curved channels.

Example 48: The method of any of examples 33 through 47, further comprising expanding an expanding device within the resection cavity, and wherein implanting the plurality of implantable leads comprises inserting each implantable lead of the plurality of implantable leads into tissue while the expanded device is expanded within the resection cavity.

Example 49: The method of any of examples 33 through 48, further includes cutting off a portion of at least one lead of the plurality of implantable leads, wherein the portion includes one or more electrodes; and implanting the at least one lead with the portion removed.

Example 50: The method of any of examples 26 through 49, wherein one or more electrodes of the plurality of electrodes comprises an electrode structure configured to conform to a surface of the resection cavity.

Example 51: A system comprising the implantable medical device configured to perform the method of any of examples 26 through 50.

Example 52: A system includes a plurality of implantable leads configured to be disposed within tissue adjacent a resection cavity of a patient; and an implantable medical device configured to deliver electric field therapy via the plurality of implantable leads disposed adjacent to the resection cavity.

Example 53: The system of example 52, further comprising an external power device configured to deliver power wirelessly to the implantable medical device.

Example 54: The system of any of examples 52 and 53, further comprising a multiplexer separate from the medical device and configured to select subsets of leads from the plurality of implantable leads for respective electric fields of the electric field therapy, wherein the multiplexer is configured to couple to the plurality of implantable leads and couple to the implantable medical device via fewer conductors than the plurality of implantable medical leads coupled to the multiplexer.

Example 55: A method includes removing a tumor from tissue to create a resection cavity; implanting a plurality of leads into the tissue adjacent to the resection cavity; affixing the plurality of leads at least one of the tissue or bone; tunneling proximal ends of the leads to an implantation pocket; and coupling the proximal ends of the leads to an implantable medical device configured to be placed within the implantation pocket, wherein the implantable medical is configured to deliver electric field therapy via the plurality of implantable leads disposed adjacent to the resection cavity.

Example 56: Any device, system, method, or computer-readable medium described or otherwise supported in the specification herein.

Example 57: A non-transitory computer-readable storage medium including instructions that, when executed, cause processing circuitry to control an implantable medical device to deliver electric field therapy according to the parameters defining the electric field therapy by at least alternating electric fields between electrodes of adjacent lead pairs of a plurality of implantable leads disposed adjacent to a resection cavity of a patient.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A system comprising:
memory configured to store parameters defining electric field therapy; and
processing circuitry configured to control an implantable medical device to deliver electric field therapy according to the parameters by at least alternating electric fields between electrodes of adjacent lead pairs of a plurality of implantable leads disposed adjacent to a resection cavity of a patient.

2. The system of claim 1, wherein the processing circuitry is configured to control the implantable medical device to deliver the electric field therapy by at least controlling the implantable medical device to deliver alternating electric fields between electrodes of each lead of the plurality of implantable leads.

3. The system of claim 1, wherein the processing circuitry is configured to control the implantable medical device to deliver the electric field therapy by at least controlling the implantable medical device to deliver alternating electric fields between electrodes of lead pairs across the resection cavity, the plurality of implantable leads comprising the lead pairs.

4. The system of claim 1, wherein the processing circuitry is configured to control the implantable medical device to interleave first alternating fields between a first lead pair across the resection cavity from each other with second alternating fields between a second lead pair across the resection cavity from each other.

5. The system of claim 1, wherein the plurality of implantable leads comprises at least six implantable leads, wherein each lead of the plurality of implantable leads comprises a plurality of electrodes.

6. The system of claim 1, wherein the processing circuitry is configured to control the implantable medical device to deliver the electric field therapy by at least controlling a multiplexer separate from the implantable medical device to select subsets of leads from the plurality of implantable leads for respective electric fields of the electric field therapy, wherein the plurality of implantable leads are coupled to the multiplexer, and wherein the multiplexer is coupled to the implantable medical device via few conductors than the plurality of implantable medical leads coupled to the multiplexer.

7. The system of claim 1, further comprising the implantable medical device, and wherein the implantable medical device is configured to receive operational power wirelessly from an external power device.

8. The system of claim 1, further comprising a spacer, wherein the plurality of implantable leads are configured to be positioned such that a first lead of the plurality of implantable leads is positioned with respect to a second lead of the plurality of implantable leads via a spacer through which the first lead is inserted, wherein the spacer comprises at least one flange extending radially outward from a channel of the spacer through which the first lead is disposed, the at least one flange extending radially outward a distance corresponding to a target distance between the first lead and the second lead.

9. The system of claim 1, further comprising primary fixation configured to secure each lead of the plurality of implantable leads to brain tissue of the patient and secondary fixation configured to secure each lead of the plurality of implantable leads to a skull of the patient.

10. The system of claim 1, further comprising the implantable medical device, wherein the implantable medical device comprises the processing circuitry.

11. A method comprising:
controlling, by processing circuitry, an implantable medical device to deliver electric field therapy according to the parameters defining the electric field therapy by at least alternating electric fields between electrodes of adjacent lead pairs of a plurality of implantable leads disposed adjacent to a resection cavity of a patient.

12. The method of claim 11, wherein controlling the implantable medical device to deliver the electric field therapy comprises controlling the implantable medical device to deliver alternating electric fields between electrodes of each lead of the plurality of implantable leads.

13. The method of claim 11, further comprising interleaving first alternating fields between a first lead pair across the resection cavity from each other with second alternating fields between a second lead pair across the resection cavity from each other.

14. The method of claim 11, wherein the plurality of implantable leads comprises at least six implantable leads, wherein each lead of the plurality of implantable leads comprises a plurality of electrodes.

15. The method of claim 11, wherein controlling the implantable medical device to deliver the electric field therapy comprises controlling a multiplexer separate from the implantable medical device to select subsets of leads from the plurality of implantable leads for respective electric fields of the electric field therapy, wherein the plurality of implantable leads are coupled to the multiplexer, and wherein the multiplexer is coupled to the implantable medical device via few conductors than the plurality of implantable medical leads coupled to the multiplexer.

16. The method of claim 11, further comprising implanting the plurality of implantable leads through at least a portion of tissue defining the resection cavity.

17. The method of claim 16, wherein implanting the plurality of implantable leads comprises implanting the plurality of implantable leads such that each electrode of each implantable lead of the plurality of implantable leads is surrounded by the tissue.

18. The method of claim 16, wherein implanting the plurality of implantable leads comprises implanting the plurality of implantable leads such that at least one electrode carried by the plurality of implantable leads is positioned within the resection cavity.

19. The method of claim 16, wherein implanting the plurality of implantable leads comprises positioning a first lead of the plurality of implantable leads with respect to a second lead of the plurality of implantable leads via a spacer through which the first lead is inserted, wherein the spacer comprises at least one flange extending radially outward from a channel of the spacer through which the first lead is disposed, the at least one flange extending radially outward a distance corresponding to a target distance between the first lead and the second lead.

20. A non-transitory computer-readable storage medium comprising instructions that, when executed, cause processing circuitry to:
control an implantable medical device to deliver electric field therapy according to the parameters defining the electric field therapy by at least alternating electric fields between electrodes of adjacent lead pairs of a plurality of implantable leads disposed adjacent to a resection cavity of a patient.
